(19) United States
(12) Patent Application Publication
(10) Pub. No.: US 2026/0182873 A1
(43) Pub. Date: Jul. 2, 2026

(54) Multimodal Wearable System for Continuous Physiological and Metabolic Monitoring Using Integrated Acoustic, Impedance, and Volatile Organic Compound Sensing with Machine Learning Pattern Classification for Disease Detection, Prediction, and Therapeutic Intervention

(71) Applicant: SHUBHAM CHANDRA, Katy, TX (US)

(72) Inventor: SHUBHAM CHANDRA, Katy, TX (US)

(73) Assignee: Shubham Chandra, Katy, TX (US)

(21) Appl. No.: 19/445,625

(22) Filed: Jan. 12, 2026

Related U.S. Application Data
(63) Continuation-in-part of application No. 18/318,719, filed on May 17, 2023.
(63) Continuation-in-part of application No. 18/932,529, filed on Oct. 30, 2024.

(51) Int. Cl. A61B 5/1477; A61B 5/00; A61B 5/02; A61B 5/0205; A61B 5/053

(52) U.S. Cl. CPC A61B 5/1477; A61B 5/0002; A61B 5/0205; A61B 5/4836; A61B 5/4866; A61B 5/7267; A61B 5/746; A61B 5/02007; A61B 5/053; A61B 5/4857; A61B 2503/40

(57) ABSTRACT

A dual-sensor bone-conduction system enables real-time theranostic monitoring of vagal, vascular, and neuro-physiological activity through simultaneous acoustic fingerprinting and bio-impedance spectroscopy. A bone-conduction transducer transmits frequency-swept mechanical energy (32 Hz-1 MHz) while a piezoelectric receiver and tetrapolar electrodes capture reflected and dielectric responses. Machine-learning fusion of these signals yields indices of vascular resonance, mechanical-electrical coherence, and bilateral autonomic symmetry. Embodiments include cranial EEG-type headsets, thoracic ECG-type modules, smartwatches, veterinary collars, and plant-stress sensors. The system supports diagnostic imaging, closed-loop acoustic neuromodulation, and cross-species health or stress detection in a low-power, wearable, and non-invasive form factor.

### FIELD OF THE INVENTION

The present invention relates to multimodal wearable monitoring systems for biomedical, veterinary, and agricultural applications. More particularly, it concerns integrated sensor architectures capable of extracting vagal, vascular, electrophysiological, and metabolic biomarkers in real time through simultaneous acoustic fingerprinting, bio-impedance modulation, and volatile organic compound (VOC) detection and classification. The invention further relates to machine learning systems for VOC pattern classification enabling disease state prediction across metabolic, neurological, psychiatric, oncological, cardiovascular, respiratory, autoimmune, infectious, and inflammatory disease categories including but not limited to diabetes, Alzheimer's disease, Parkinson's disease, epilepsy, post-traumatic stress disorder, cancer, sepsis, heart failure, chronic obstructive pulmonary disease, inflammatory bowel disease, and systemic infections. The invention additionally relates to plant physiological monitoring through acoustic-impedance-VOC sensing for agricultural stress detection and environmental monitoring applications.

### BACKGROUND OF THE INVENTION

### A. The Autonomic Nervous System: Structure, Function, and Clinical Significance

The autonomic nervous system (ANS) constitutes the division of the peripheral nervous system responsible for regulating involuntary physiological processes essential for homeostasis and survival. These processes include cardiac function, blood pressure regulation, respiration, digestion, thermoregulation, urinary function, sexual function, and immune modulation. The ANS operates continuously without conscious control, adjusting physiological parameters in response to internal states and environmental demands.

The ANS comprises two primary divisions with generally opposing effects. The sympathetic nervous system (SNS) mediates the “fight-or-flight” response, preparing the organism for physical exertion or threat response through increased heart rate, elevated blood pressure, bronchodilation, pupil dilation, reduced digestive activity, and mobilization of energy stores. Sympathetic preganglionic neurons originate in the thoracolumbar spinal cord (T1-L2) and synapse in paravertebral or prevertebral ganglia; postganglionic neurons release norepinephrine onto target organs. The parasympathetic nervous system (PNS) mediates “rest-and-digest” functions, promoting energy conservation and restorative processes through reduced heart rate, enhanced digestive activity, and tissue repair. Parasympathetic preganglionic neurons originate in cranial nerve nuclei (III, VII, IX, X) and sacral spinal cord (S2-S4); postganglionic neurons release acetylcholine onto target organs.

The vagus nerve (cranial nerve X) serves as the primary parasympathetic pathway, carrying approximately 75-80% of all parasympathetic efferent fibers in the body. The term “vagus” derives from Latin meaning “wandering,” aptly describing the nerve's extensive anatomical distribution. The vagus originates in the medulla oblongata from the nucleus ambiguus (motor functions), dorsal motor nucleus (parasympathetic functions), and nucleus tractus solitarius (sensory functions). It exits the skull through the jugular foramen, descends through the neck within the carotid sheath alongside the internal jugular vein and carotid artery, and distributes branches to pharynx, larynx, heart, lungs, esophagus, stomach, intestines, liver, and other abdominal viscera.

Critically, approximately 80% of vagal fibers are afferent (sensory), carrying information from visceral organs to the brainstem. These afferent signals inform central autonomic regulation, enabling the brain to monitor and respond to peripheral physiological states. Vagal afferents transmit information about gastric distension, intestinal nutrient content, inflammatory mediators, microbial metabolites, and cardiopulmonary status. This bidirectional communication—termed the “vagal afferent pathway”—is fundamental to gut-brain communication, immune regulation, and metabolic control.

Vagal tone refers to the degree of parasympathetic influence on physiological function at any given time. Vagal tone is not a fixed property but fluctuates continuously in response to breathing, posture, emotional state, cognitive demands, and circadian rhythms. High vagal tone indicates robust parasympathetic activity and is associated with cardiovascular resilience, low resting heart rate, high heart rate variability, effective emotional regulation, reduced systemic inflammation, and cognitive flexibility. Low vagal tone indicates parasympathetic withdrawal or relative sympathetic dominance, associated with elevated resting heart rate, reduced heart rate variability, cardiovascular risk, chronic inflammation, mood disorders, and cognitive impairment.

The clinical significance of vagal function extends across virtually all organ systems. Cardiovascular effects include heart rate reduction through sinoatrial node inhibition, atrioventricular conduction slowing, and modest negative inotropy. Respiratory effects include bronchoconstriction and mucus secretion. Gastrointestinal effects include increased motility, secretion, and sphincter relaxation. Immune effects—mediated through the cholinergic anti-inflammatory pathway—include suppression of pro-inflammatory cytokine release. Metabolic effects include modulation of hepatic glucose production and pancreatic insulin secretion. Neuropsychiatric effects include mood regulation, stress resilience, and cognitive function. Present-day technologies measure vagal tone only indirectly through heart rate variability (HRV) analysis. HRV quantifies the variation in time intervals between consecutive heartbeats (R-R intervals on electrocardiogram). High-frequency HRV power (0.15-0.4 Hz), corresponding to respiratory frequencies, primarily reflects parasympathetic modulation of sinoatrial node firing rate through respiratory sinus arrhythmia—the phenomenon of heart rate acceleration during inspiration and deceleration during expiration. Low-frequency HRV power (0.04-0.15 Hz) reflects both sympathetic and parasympathetic influences, with interpretation remaining controversial. Time-domain HRV metrics include SDNN (standard deviation of R-R intervals), RMSSD (root mean square of successive differences), and pNN50 (percentage of successive intervals differing by >50 ms).

However, HRV-based vagal assessment suffers fundamental limitations. First, HRV represents downstream cardiac effects of autonomic modulation rather than direct vagal nerve activity, introducing measurement indirection. Second, HRV analysis typically requires 5-minute or longer recording windows for frequency-domain analysis, precluding real-time assessment. Third, HRV measurements are highly susceptible to motion artifacts, limiting ambulatory application. Fourth, HRV cannot distinguish between reduced vagal outflow and increased sympathetic activity, as both produce similar HRV patterns. Fifth, HRV provides no information about vagal effects on non-cardiac organs. The present invention overcomes these limitations through direct acoustic-mechanical interrogation of vagal-mediated vascular dynamics.

### B. Volatile Organic Compounds: Biochemical Origins, Physiological Significance, and Disease Biomarkers

Volatile organic compounds (VOCs) are carbon-containing molecules characterized by high vapor pressure at ambient temperature, enabling their existence in gaseous phase under physiological conditions. The defining characteristic is volatility—the tendency to evaporate—which allows VOCs to be detected in exhaled breath, emanate from skin, and be released from biological fluids. VOCs range from simple molecules (methane, CH4; molecular weight 16 Da) to complex structures (terpenes, sesquiterpenes; molecular weights 150-250 Da). Human metabolism generates thousands of distinct VOCs through diverse biochemical pathways. Endogenous VOC sources include:

Cellular respiration and energy metabolism: Glucose oxidation through glycolysis and the citric acid cycle produces carbon dioxide and water as primary products, but incomplete oxidation and alternative pathway utilization generate volatile intermediates. Acetone (propan-2-one, CH3COCH3) arises from spontaneous decarboxylation of acetoacetate during ketogenesis when fatty acid oxidation exceeds acetyl-CoA utilization capacity. Acetone concentration correlates with ketone body levels and serves as a biomarker for diabetic ketoacidosis, fasting, ketogenic diet adherence, and metabolic stress.

Lipid peroxidation: Reactive oxygen species (ROS) attack polyunsaturated fatty acids in cellular membranes, initiating chain reactions that fragment lipid molecules into volatile aldehydes. Malondialdehyde (MDA), 4-hydroxynonenal (4-HNE), hexanal, heptanal, octanal, and nonanal are characteristic lipid peroxidation products. These aldehydes serve as oxidative stress biomarkers elevated in cancer, neurodegeneration, cardiovascular disease, and inflammatory conditions. The specific aldehyde profile depends on the fatty acid composition of peroxidized membranes, potentially providing tissue-of-origin information.

Protein and amino acid catabolismamino acid degradation generates volatile nitrogenous compounds. Ammonia (NH3) arises from deamination reactions and is normally converted to urea in the hepatic urea cycle; elevated blood and tissue ammonia indicates hepatic dysfunction or overwhelming protein catabolismrimethylamine (TMA) is produced by gut bacterial metabolism of dietary choline, carnitine, and betaine; trimethylamine N-oxide (TMAO) formed by hepatic oxidation of TMA is associated with cardiovascular disease risk. Indole and skatole (3-methylindole) derive from bacterial tryptophan metabolism and serve as gut dysbiosis markers.

Microbial metabolism: The intestinal microbiome-comprising approximately 10\hat{ }13-10\hat{ }14 bacterial cells representing over 1,000 species-produces abundant volatile metabolites through fermentation of dietary substrates and host-derived compounds. Short-chain fatty acids (SCFAs)—acetate (C2), propionate (C3), and butyrate (C4)—are primary fermentation products of dietary fiber, serving as colonocyte energy sources, signaling molecules, and immune modulators. Hydrogen sulfide (H2S) is produced by sulfate-reducing bacteria from dietary and endogenous sulfur sources. Methane (CH4) is produced by methanogenic archaea. Volatile phenolic compounds (phenol, p-cresol) arise from bacterial tyrosine metabolism. The collective volatile metabolome reflects microbiome composition and metabolic activity, changing with diet, disease, and antibiotic exposure.

Hepatic biotransformation: The liver metabolizes endogenous compounds and xenobiotics through Phase I (oxidation, reduction, hydrolysis) and Phase II (conjugation) reactions. Cytochrome P450 enzymes introduce or expose functional groups, often generating volatile intermediates. Hepatic dysfunction alters biotransformation capacity, producing characteristic VOC signatures. Dimethyl sulfide and mercaptans accumulate in hepatic encephalopathy. Altered limonene metabolism has been associated with liver cirrhosis.

Tissue necrosis and cellular breakdown: Cell death releases intracellular contents including volatile compounds normally sequestered within membranes. Tumor necrosis produces distinctive VOC signatures. Ischemic tissue generates volatile markers of anaerobic metabolism.

Inflammatory mediator metabolism: Cyclooxygenase and lipoxygenase pathways metabolize arachidonic acid to prostaglandins, leukotrienes, and other eicosanoids, generating volatile byproducts. Isoprene (2-methyl-1,3-butadiene) is produced through the mevalonate pathway and may reflect cholesterol synthesis and inflammatory activity. Ethane and pentane are produced by lipid peroxidation during inflammation.

Exogenous VOC sources include inhaled environmental compounds, ingested substances, dermal absorption, and microbial metabolism of xenobiotics. These exogenous VOCs may confound endogenous biomarker analysis and require consideration in pattern classification algorithms.

### C. Skin as a VOC Emission Surface: Anatomy, Physiology, and Measurement Considerations

Human skin provides continuous VOC emission accessible to non-invasive wearable sensing. The skin is the body's largest organ (approximately 2 m² surface area in adults), serving as a barrier, thermoregulatory organ, sensory interface, immune organ, and excretory surface. Three primary pathways deliver VOCs to the skin surface:

Eccrine perspiration: Approximately 2-4 million eccrine sweat glands distributed across nearly all body surfaces secrete hypotonic sweat containing water, electrolytes, urea, lactate, and trace amounts of other metabolites. VOCs dissolved in blood partition into sweat based on their blood-sweat partition coefficients, which depend on lipophilicity, molecular size, and protein binding. Sweat reaches the skin surface through eccrine ducts, where evaporation releases dissolved VOCs into the headspace. Eccrine sweating is controlled primarily by sympathetic cholinergic innervation responding to thermal and emotional stimuli. Sweat rate varies from minimal insensible perspiration at rest to over 2 L/hour during intense exercise or heat exposure.

Sebaceous secretion: Sebaceous glands, associated with hair follicles and concentrated on face, scalp, and upper trunk, secrete sebum—a complex lipid mixture comprising triglycerides, wax esters, squalene, and free fatty acids. Sebum provides waterproofing, antimicrobial activity, and pheromone delivery. Sebaceous lipids undergo oxidation (generating volatile aldehydes) and bacterial metabolism (generating volatile fatty acids and other compounds) on the skin surface. Sebum composition varies with age, sex, diet, and disease states. Notably, Parkinson's disease alters sebum composition, producing detectable VOC signatures years before motor symptom onset.

Transcutaneous diffusion: Highly volatile, lipophilic compounds diffuse directly through the stratum corneum—the outermost layer of dead, keratinized epithelial cells—from dermal capillaries. Diffusion rate depends on compound volatility, lipophilicity, molecular size, skin temperature, and stratum corneum thickness and hydration. This pathway provides continuous emission independent of sweating.

Factors affecting skin VOC emission include: body site (sebaceous gland density, skin thickness, blood flow); temperature (emission increases approximately 10% per° C.); humidity (affecting sweat evaporation); activity level (affecting blood flow and sweating); circadian rhythm (core body temperature and hormonal cycles); diet (directly affecting VOC production); medications (altering metabolism); topical products (introducing exogenous VOCs); and individual variation (genetics, microbiome).

### D. The Gut-Brain-Immune Axis: Mechanisms, Dysregulation, and Disease Implications

The gut-brain axis constitutes a bidirectional communication network integrating the gastrointestinal tract, enteric nervous system, central nervous system, and immune system. This axis operates through multiple parallel pathways:

Neural pathway: The vagus nerve provides the primary neural connection between gut and brain. Vagal afferents terminating in the intestinal mucosa sense mechanical distension, nutrient content, pH, osmolarity, and chemical signals including microbial metabolites, cytokines, and hormones. These signals travel to the nucleus tractus solitarius in the brainstem, which projects to hypothalamus, limbic system, and cortex, influencing appetite, mood, and behavior. Vagal efferents modulate gut motility, secretion, blood flow, and immune function. The enteric nervous system—sometimes called the “second brain”—contains approximately 500 million neurons capable of autonomous gut function regulation while also communicating bidirectionally with the CNS.

Endocrine pathway: Enteroendocrine cells scattered throughout the gut epithelium produce hormones in response to luminal contents. Cholecystokinin (CCK), glucagon-like peptide-1 (GLP-1), peptide YY (PYY), ghrelin, and other gut hormones regulate appetite, insulin secretion, and metabolism while also influencing brain function directly through blood-brain barrier-crossing or indirectly through vagal afferent signaling.

Immune pathway: The gut-associated lymphoid tissue (GALT) represents the largest immune organ in the body, continuously sampling luminal contents and maintaining tolerance to commensal organisms while defending against pathogens. Intestinal immune activation produces cytokines (IL-1β, IL-6, TNF-α) that enter systemic circulation and influence brain function through direct transport across the blood-brain barrier, vagal afferent signaling, and activation of brain endothelial cells producing central inflammatory mediators.

Microbial metabolite pathway: Gut bacteria produce neuroactive compounds that influence brain function through multiple mechanisms. Neurotransmitter production: gut bacteria synthesize approximately 95% of the body's serotonin, along with dopamine, norepinephrine, GABA, and acetylcholine. While bacterial neurotransmitters may not cross the blood-brain barrier directly, they influence enteric nervous system function and vagal afferent signaling. Short-chain fatty acids (SCFAs): acetate, propionate, and butyrate produced by fiber fermentation serve as energy sources (butyrate for colonocytes; acetate and propionate absorbed systemically), signaling molecules (activating G-protein-coupled receptors GPR41, GPR43 on immune cells, enteroendocrine cells, and neurons), and epigenetic modulators (butyrate as histone deacetylase inhibitor). SCFAs influence blood-brain barrier integrity, microglial activation, and neuroinflammation. Tryptophan metabolites: gut bacteria metabolize dietary tryptophan through multiple pathways, producing indole derivatives, kynurenine pathway metabolites, and serotonin. These metabolites influence immune function, gut barrier integrity, and brain function.

Gut dysbiosis—defined as alterations in intestinal microbial community composition, diversity, or metabolic activity associated with disease—disrupts gut-brain axis function through multiple mechanisms. Reduced microbial diversity limits metabolic capacity and resilience. Pathobiont overgrowth (expansion of potentially pathogenic species) promotes inflammation and barrier dysfunction. Reduced beneficial species (particularly butyrate-producing Firmicutes) decreases anti-inflammatory signaling and epithelial nutrition. Altered metabolite production changes the chemical milieu sensed by the host.

Dysbiosis-associated VOC signatures reflect these microbial metabolic changes and are detectable through skin emission. Characteristic patterns include:

Elevated indoles (indole, skatole, indole-3-acetate): Produced by bacterial tryptophan metabolism, particularly by proteolytic species. Elevated in inflammatory bowel disease, small intestinal bacterial overgrowth, and hepatic encephalopathy.

Elevated phenolic compounds (phenol, p-cresol, p-cresyl sulfate): Produced by bacterial tyrosine metabolism. Elevated in chronic kidney disease (where uremic toxin accumulation occurs), autism spectrum disorder, and IBD.

Elevated hydrogen sulfide: Produced by sulfate-reducing bacteria. Associated with ulcerative colitis and potentially colorectal cancer.

Altered SCFA ratios: Reduced butyrate relative to acetate and propionate indicates decreased beneficial fermenters. Elevated branched-chain fatty acids (isobutyrate, isovalerate) indicate increased protein fermentation.

Elevated trimethylamine: Produced from dietary choline/carnitine metabolism. Associated with cardiovascular disease risk through TMAO pathway.

### E. Disease Detection Through VOC Pattern Analysis: Evidence and Applications

Extensive research demonstrates that specific diseases produce characteristic VOC signatures detectable in breath, skin emanations, and other biological samples. Key evidence includes:

Cancer detection: Multiple studies demonstrate that trained dogs can detect various cancers (lung, breast, colorectal, ovarian, prostate, melanoma) in breath or urine samples with sensitivity and specificity often exceeding 90%. Electronic nose studies have identified cancer-specific VOC patterns. Lung cancer produces elevated aldehydes (hexanal, heptanal), benzene derivatives, and alkanes. Colorectal cancer produces elevated indole, methylindole, and short-chain fatty acids. Breast cancer produces elevated alkanes and methylated alkanes. The underlying mechanisms include altered tumor metabolism (Warburg effect producing lactate and volatile byproducts), oxidative stress (lipid peroxidation products), and immune response (inflammatory VOCs).

Infectious disease detection: Bacterial, viral, and fungal infections produce characteristic VOC signatures reflecting pathogen metabolism and host response. Pseudomonas aeruginosa produces 2-aminoacetophenone. Mycobacterium tuberculosis produces methyl nicotinate. Clostridium difficile produces p-cresol and isocaproic acid. COVID-19 has been associated with specific breath VOC patterns. Sepsis—systemic inflammatory response to infection—produces elevated acetone, 2-butanone, isoprene, and other markers reflecting metabolic derangement and oxidative stress.

Metabolic disease detection: Diabetes produces elevated acetone and isopropanol (from ketone body metabolism). Diabetic ketoacidosis produces markedly elevated acetone (>2 ppm in skin emission). Chronic kidney disease produces elevated ammonia, trimethylamine, and dimethylamine. Liver disease produces elevated dimethyl sulfide, mercaptans, and limonene. Phenylketonuria produces elevated phenylacetic acid.

Neurological disease detection: Parkinson's disease produces characteristic sebum VOC patterns detectable by trained humans (“musky” odor) and analytical methods—hippuric acid, eicosane, and octadecanal—potentially years before motor symptom onset. Alzheimer's disease produces elevated lipid peroxidation aldehydes reflecting neuronal membrane damage. Epilepsy produces pre-ictal VOC changes (menthone, isopulegol, linalyl acetate) detectable by seizure-alert dogs.

Psychiatric disease detection: Schizophrenia has been associated with altered VOC profiles including pentane and carbon disulfide. Major depression correlates with inflammatory VOC markers. PTSD produces altered stress hormone metabolites and inflammatory markers detectable in VOC profiles.

Inflammatory and autoimmune diseases: Inflammatory bowel disease produces elevated indoles, sulfur compounds, and inflammatory VOC markers. Rheumatoid arthritis produces elevated pentane and other oxidative stress markers. Asthma produces elevated nitric oxide and inflammatory eicosanoid metabolites.

### F. Sepsis: Pathophysiology and VOC Biomarkers

Sepsis represents life-threatening organ dysfunction caused by dysregulated host response to infection. Sepsis affects approximately 49 million people annually worldwide, causing 11 million deaths—nearly 20% of all global deaths. Early detection and treatment are critical: each hour of delayed antibiotic administration increases mortality by approximately 7.6%. The pathophysiology of sepsis involves complex interactions between pathogen and host: Initial infection triggers pattern recognition receptor (PRR) activation: Toll-like receptors, NOD-like receptors, and other PRRs recognize pathogen-associated molecular patterns (PAMPs) including lipopolysaccharide (LPS, gram-negative bacteria), lipoteichoic acid (gram-positive bacteria), flagellin, and microbial nucleic acids.

PRR activation initiates inflammatory cascade: NF-κB and other transcription factors drive production of pro-inflammatory cytokines (TNF-α, IL-1β, IL-6), chemokines, and adhesion molecules. Neutrophil activation, complement activation, and coagulation cascade activation follow.

Dysregulated inflammation causes collateral damage: Excessive cytokine release (“cytokine storm”) causes endothelial dysfunction, capillary leak, vasodilation, and tissue edema. Microcirculatory failure impairs oxygen delivery. Coagulopathy produces disseminated intravascular coagulation. Multi-organ dysfunction syndrome (MODS) ensues.

Metabolic derangement accompanies sepsis: Increased energy demands, impaired substrate utilization, and mitochondrial dysfunction produce characteristic metabolic patterns. Lactate accumulates from anaerobic metabolism and impaired clearance. Ketogenesis increases. Protein catabolismaccelerates. Oxidative stress intensifies.

These pathophysiological processes produce characteristic VOC signatures: Acetone: Elevated from increased ketogenesis and metabolic stress.

2-Butanone (methyl ethyl ketone): Elevated in sepsis, possibly from bacterial metabolism or altered hepatic function.

Isoprene: Altered levels reflecting cholesterol synthesis changes and oxidative stress.

Pentane and ethane: Elevated from lipid peroxidation during oxidative stress.

Hydrogen sulfide: May be elevated from bacterial metabolism and tissue hypoxia.

Ammonia: Elevated from protein catabolism and potential hepatic dysfunction.

Aldehydes: Elevated from lipid peroxidation and cellular damage.

Autonomic dysfunction is a hallmark of sepsis. Sepsis produces profound vagal withdrawal with relative sympathetic dominance, manifested as reduced heart rate variability, elevated resting heart rate, and impaired baroreflex sensitivity. This autonomic dysfunction contributes to cardiovascular instability, immune dysregulation (loss of cholinergic anti-inflammatory pathway), and gut barrier dysfunction.

The present invention's multimodal approach—combining sepsis-associated VOC patterns with autonomic biomarkers (particularly MECI reflecting vagal tone)—provides a powerful early sepsis detection capability. The Sepsis Risk Index (SRI) integrates:
Inflammatory VOC markers (isoprene, pentane, aldehydes)
Metabolic stress markers (acetone, 2-butanone)
Autonomic dysfunction (declining MECI, elevated VRSI)
Impedance changes reflecting tissue edema

### G. Cancer: Metabolic Alterations and VOC Biomarkers

Cancer comprises over 200 distinct diseases characterized by uncontrolled cell proliferation, but common metabolic alterations produce overlapping VOC signatures. The Warburg effect—preferential glycolysis even under aerobic conditions—represents a near-universal cancer metabolic phenotype producing elevated lactate and associated volatile metabolites.

Cancer-associated VOC alterations arise from multiple mechanisms:

Altered primary metabolism: The Warburg effect increases glycolytic flux, producing lactate and volatile byproducts. Glutaminolysis—increased glutamine catabolism—generates ammonia and other nitrogen-containing volatiles. Increased lipid synthesis for membrane production alters fatty acid metabolism.

Oxidative stress: Cancer cells exhibit elevated reactive oxygen species (ROS) from mitochondrial dysfunction, oncogene activation, and rapid proliferation. ROS attack membrane lipids, producing characteristic aldehyde patterns (hexanal, heptanal, octanal, nonanal) through lipid peroxidation.

Inflammation: Tumor-associated inflammation produces elevated inflammatory VOC markers including isoprene, pentane, and ethane.

Tissue necrosis: Tumor necrosis releases intracellular contents and generates volatile degradation products.

Altered microbiome: Cancer and cancer treatment alter gut microbiome composition, changing microbial VOC production.

Tumor-specific metabolism: Certain tumors exhibit characteristic metabolic patterns.

Hepatocellular carcinoma produces elevated dimethyl sulfide. Colorectal cancer produces elevated indole derivatives reflecting gut microbiome alterations.

Specific cancer VOC signatures identified in research include:

Lung cancer: Elevated hexanal, heptanal, octanal, nonanal (aldehydes from lipid peroxidation); benzene derivatives; alkanes (pentane, hexane); and isoprene.

Breast cancer: Elevated alkanes (nonane, decane); methylated alkanes; and formaldehyde.

Colorectal cancer: Elevated indole, skatole, p-cresol (reflecting dysbiosis); short-chain fatty acids; and aldehydes.

Gastric cancer: Elevated 2-propenenitrile, 2-butoxy-ethanol, and furfural.

Liver cancer: Elevated hexanal, 1-octen-3-ol, and octane.

Prostate cancer: Altered isovaleric acid, methanol, and acetone patterns.

The Oncological Risk Index (ORI) computed by the present invention integrates cancer-associated VOC patterns with inflammatory biomarkers and autonomic dysfunction indicators to provide continuous cancer screening capability.

### DESCRIPTION OF RELATED ART

### A. Wearable Biofluid Sensors

U.S. Patent Application Publication No. 2017/0325724 (Wang et al.) discloses non-invasive wearable electrochemical sensors utilizing iontophoresis for interstitial fluid extraction and analyte detection. This reference is limited to electrochemical detection of specific analytes and does not teach volatile organic compound detection, pattern classification, acoustic sensing, or integration with autonomic biomarkers.

### B. Electronic Nose and Gas Sensor Arrays

Laboratory electronic nose (e-nose) systems employ metal-oxide semiconductor (MOX) sensor arrays for VOC detection and pattern recognition. Commercial systems including the Cyranose 320, PEN3, and zNose have demonstrated disease detection capabilities in research settings. However, these systems are designed for laboratory use, lack wearable form factors, do not incorporate autonomic biomarker measurement, and do not provide closed-loop therapeutic intervention capability.

### C. Bone Conduction and Acoustic Wearable Devices

U.S. Pat. No. 9,682,001 discloses a wearable bone conduction device for vestibular therapy and sympathetic arousal monitoring. This reference uses bone conduction solely for audio delivery and vestibular stimulation rather than diagnostic tissue interrogation. The reference lacks bio-impedance measurement, VOC detection, and disease-specific biomarker computation. U.S. Pat. No. 11,235,156 discloses a wearable audio device with vagus nerve stimulation triggered by detected physiological events. This reference provides stimulation without comprehensive diagnostic sensing, VOC detection, or multimodal pattern classification.

### D. Breath Analysis Systems

Research and commercial breath analyzers detect VOCs for specific applications including alcohol detection (breathalyzers), diabetes monitoring (acetone), and Helicobacter pylori detection (urea breath test). However, breath analysis requires active sampling maneuvers, cannot provide continuous monitoring, and does not capture skin-specific VOC emission patterns that may differ from breath composition.

### E. Sepsis Detection Systems

Current sepsis detection relies on clinical assessment, vital signs (heart rate, blood pressure, respiratory rate, temperature), laboratory biomarkers (lactate, procalcitonin, C-reactive protein), and culture results. These approaches suffer from delayed detection (laboratory turnaround time), lack of specificity (vital sign changes occur late), or invasiveness (blood draws). No prior art teaches continuous non-invasive sepsis detection combining VOC pattern classification with autonomic biomarkers.

### F. Cancer Screening Approaches

Current cancer screening relies on imaging modalities (mammography, CT, colonoscopy), tissue sampling (biopsy), and specific biomarker assays (PSA, CA-125, CEA). These approaches are performed episodically rather than continuously, may be invasive, and are limited to specific cancer types. While research demonstrates VOC-based cancer detection feasibility, no wearable continuous cancer screening system exists.

### G. Deficiencies in the Prior Art

No prior art, alone or in combination, teaches or suggests: (1) a wearable system combining bone-conduction acoustic tissue interrogation with bio-impedance spectroscopy and VOC pattern classification; (2) continuous computation of vagal and autonomic biomarkers (VRSI, MECI, BASI, RVCF) from mechanical-electrical tissue measurements; (3) VOC pattern classification for disease detection spanning metabolic, neurological, psychiatric, oncological, cardiovascular, respiratory, autoimmune, and infectious disease categories; (4) integration of autonomic biomarkers with classified VOC patterns for multimodal disease prediction; (5) closed-loop therapeutic intervention triggered by detected disease states; or (6) cross-species applicability of a unified sensing framework.

### SUMMARY OF THE INVENTION

The present invention provides a multimodal wearable monitoring system comprising:
(a) A bone-conduction transducer configured to emit frequency-swept acoustic energy through biological tissue spanning frequencies from 32 Hz to 1 MHz, and to receive reflected mechanical responses enabling characterization of tissue mechanical properties and vascular dynamics;
(b) A piezoelectric or micro-electromechanical systems (MEMS) sensor coupled to said transducer for high-resolution detection of mechanical vibrations, tissue resonance characteristics, and acoustic transmission properties;
(c) A tetrapolar bio-impedance module comprising four electrodes configured to inject alternating micro-currents at levels safe for continuous use (≤10 μA rms) and measure complex tissue impedance magnitude and phase across frequencies from 10 Hz to 1 MHz;
(d) A metal-oxide semiconductor (MOX) volatile organic compound (VOC) sensor array comprising multiple sensor elements with differential chemical sensitivity, positioned to continuously detect and classify volatile organic compound patterns from skin emissions;
(e) A processor configured to: execute time-frequency analysis of acoustic and impedance signals to generate autonomic and vascular biomarkers; perform machine learning classification of VOC sensor array responses to identify disease-associated patterns; fuse multimodal data (acoustic, impedance, VOC) using deep learning architectures for integrated disease state prediction; and compute composite health indices;
(f) A communication interface for transmitting processed biomarkers, health assessments, and alerts to external displays, mobile applications, cloud-based analytics platforms, and healthcare provider systems; and
(g) Optional closed-loop therapeutic stimulation capability providing acoustic neuromodulation triggered by detected autonomic dysfunction or disease states.

### Definitions and Terminology

The following definitions apply throughout this specification:

Acoustic Fingerprinting: The process of characterizing biological tissue mechanical properties through analysis of acoustic wave propagation, reflection, attenuation, and resonance patterns generated by frequency-swept mechanical excitation. Different tissues exhibit characteristic acoustic impedance, attenuation coefficients, and resonance frequencies that change with physiological state, enabling non-invasive tissue characterization.

Autonomic Nervous System (ANS): The division of the peripheral nervous system regulating involuntary physiological processes including cardiac function, blood pressure, respiration, digestion, and immune function. Comprises sympathetic (fight-or-flight) and parasympathetic (rest-and-digest) divisions.

Bio-impedance: The opposition of biological tissue to alternating electrical current flow, comprising resistive and reactive components. Complex impedance Z=R+jX, where R is resistance (energy dissipation) and X is reactance (energy storage). Impedance varies with measurement frequency, tissue composition, hydration status, blood flow, and cellular integrity.

Biomarker: A measurable indicator of biological state or condition. Biomarkers may be molecular (blood glucose), physiological (heart rate), anatomical (tumor size), or functional (cognitive score).

Cholinergic Anti-inflammatory Pathway: A vagal efferent mechanism wherein acetylcholine release suppresses pro-inflammatory cytokine production through activation of α7 nicotinic acetylcholine receptors on immune cells, particularly macrophages.

Closed-Loop Therapeutic Intervention: Automated therapeutic delivery triggered by real-time physiological measurements, with continuous feedback enabling parameter adjustment. Contrasts with open-loop therapy delivered on fixed schedules regardless of physiological state.

Eccrine Gland: Sweat gland distributed across nearly all body surfaces, secreting hypotonic sweat in response to thermal or emotional stimuli. Primary mechanism for thermoregulation and a major pathway for VOC skin emission.

Electrodermal Activity (EDA): Electrical properties of skin resulting from sweat gland activity, measured as skin conductance (GSR) or skin potential. Reflects sympathetic nervous system activation.

Feature Extraction: The process of computing relevant characteristics from raw sensor data for use in classification algorithms. Features may include statistical measures (mean, variance), spectral properties (frequency content), temporal dynamics (rate of change), and derived indices.

Gut-Brain Axis: The bidirectional communication network between the gastrointestinal tract and central nervous system, operating through neural (vagal), endocrine (gut hormones), immune (cytokines), and microbial (metabolites) pathways.

Gut Dysbiosis: Alteration in intestinal microbial community composition, diversity, or metabolic activity associated with disease states. Characterized by reduced beneficial species, pathobiont expansion, altered metabolite production, and compromised barrier function.

Heart Rate Variability (HRV): Variation in time intervals between consecutive heartbeats, reflecting autonomic modulation of cardiac function. High-frequency HRV (0.15-0.4 Hz) primarily reflects parasympathetic activity; low-frequency HRV (0.04-0.15 Hz) reflects mixed sympathetic and parasympathetic influences.

Lipid Peroxidation: Oxidative degradation of lipids, particularly polyunsaturated fatty acids in cellular membranes, by reactive oxygen species. Produces volatile aldehydes (malondialdehyde, 4-hydroxynonenal, hexanal, nonanal) serving as oxidative stress biomarkers.

Machine Learning: Computational methods enabling systems to learn patterns from data without explicit programming. Supervised learning trains on labeled examples; unsupervised learning discovers structure in unlabeled data; reinforcement learning optimizes through trial and error.

MEMS (Micro-Electromechanical Systems): Miniaturized mechanical and electromechanical devices fabricated using integrated circuit manufacturing techniques. MEMS accelerometers, gyroscopes, and microphones enable compact motion and acoustic sensing.

Metal-Oxide Semiconductor (MOX) Sensor: A gas sensor wherein target gas molecules adsorb onto a heated metal-oxide surface (e.g., SnO₂, WO₃, ZnO, In₂O₃), changing electrical conductivity proportionally to gas concentration. Temperature modulation enables selectivity enhancement through differential response kinetics.

Microbiome: The collective genomes of microorganisms (bacteria, archaea, fungi, viruses) inhabiting a particular environment. The human gut microbiome contains approximately 10\hat{ }13-10\hat{ }14 bacterial cells representing over 1,000 species.

Pattern Classification: Assignment of input data to predefined categories based on learned features. Classification algorithms include support vector machines, random forests, neural networks, and deep learning architectures.

Pre-ictal: The period preceding seizure (ictal) onset, characterized by physiological changes potentially detectable before clinical seizure manifestation. Duration varies from minutes to hours.

Sebaceous Gland: Skin gland associated with hair follicles, secreting sebum (complex lipid mixture) providing waterproofing, antimicrobial activity, and VOC emission. Concentrated on face, scalp, and upper trunk.

Sepsis: Life-threatening organ dysfunction caused by dysregulated host response to infection. Characterized by systemic inflammation, hemodynamic instability, metabolic derangement, and multi-organ failure risk.

Short-Chain Fatty Acids (SCFAs): Fatty acids with fewer than six carbon atoms—primarily acetate (C2), propionate (C3), and butyrate (C4)—produced by bacterial fermentation of dietary fiber. SCFAs serve as energy sources, signaling molecules, and immune modulators.

Spectral Fingerprint: The characteristic multi-dimensional sensor response pattern produced by a specific analyte or analyte mixture. In MOX sensor arrays, temperature modulation produces response curves whose shape encodes mixture composition.

Stratum Corneum: The outermost layer of the epidermis, comprising dead, keratinized epithelial cells. Provides barrier function while permitting transcutaneous VOC diffusion.

Tetrapolar Configuration: An electrode arrangement using four electrodes-two for current injection (drive electrodes) and two for voltage measurement (sense electrodes)—that eliminates electrode-skin contact impedance from the measurement, improving accuracy.

Vagal Tone: The degree of parasympathetic nervous system influence on physiological function, mediated primarily through the vagus nerve. Fluctuates continuously with breathing, posture, emotional state, and circadian rhythm.

Vagus Nerve: Cranial nerve X, the primary parasympathetic nerve carrying approximately 75-80% of all parasympathetic fibers. Provides bidirectional communication between brainstem and thoracic/abdominal viscera.

Volatile Organic Compound (VOC): Carbon-containing molecule with high vapor pressure at ambient temperature, existing partially or entirely in gaseous phase under physiological conditions. VOCs are produced through metabolic processes, microbial activity, and environmental exposure.

### Computed Biomarker Indices

The processor computes the following biomarker indices from integrated sensor data:

### A. Autonomic and Vascular Biomarkers

Vascular Resonance Shift Index (VRSI): Quantifies changes in arterial wall mechanical properties. Arterial walls exhibit characteristic resonance frequencies (typically 40-180 Hz) determined by wall stiffness, vessel diameter, wall thickness, and transmural pressure. The VRSI is computed as:

$$VRSI = Δ f\_res / f\_base$$

where Δf_res represents the shift in dominant mechanical resonance frequency from an established personalized baseline f_base. Increased arterial stiffness (arteriosclerosis, hypertension, aging) elevates resonance frequency, producing positive VRSI. Vasodilation (relaxation, medication effects) reduces resonance frequency, producing negative VRSI. VRSI changes precede blood pressure elevation by months to years, providing early cardiovascular risk indication.

Mechanical-Electrical Coherence Index (MECI): Quantifies the coupling between vascular mechanical dynamics and tissue electrical properties, reflecting integrated autonomic regulation. MECI is computed as:

$$MECI = {\vert S\_ME ( f ) \vert}^{2} / [ S\_MM ( f ) \times S\_EE ( f ) ]$$

where S_ME(f) is the cross-spectral density between mechanical (acoustic) and electrical (impedance) signals, S_MM(f) is mechanical auto-spectral density, and S_EE(f) is electrical auto-spectral density. This formulation represents coherence—the frequency-domain equivalent of correlation coefficient—bounded between 0 and 1.

High MECI (>0.7) indicates that mechanical and electrical tissue dynamics are tightly coupled, reflecting intact autonomic regulation wherein vagal and sympathetic influences produce coordinated responses across modalities. Low MECI (<0.5) indicates decoupled dynamics, suggesting autonomic dysfunction, neuropathy, or severe illness. MECI declines in sepsis, heart failure, diabetic neuropathy, and other conditions affecting autonomic function.

Bilateral Autonomic Symmetry Index (BASI): Quantifies asymmetry between bilateral sensor positions, enabling detection of unilateral pathology. BASI is computed as:

$$BASI = \vert M\_L ( f ) - M\_R ( f ) \vert / \vert M\_L ( f ) + M\_R ( f ) \vert$$

where M_L(f) and M_R(f) represent mechanical spectral magnitudes from left and right sensor positions. Healthy autonomic function produces symmetric bilateral responses (BASI<0.10). Elevated asymmetry (BASI>0.15) suggests unilateral neural deficits (stroke, peripheral neuropathy, radiculopathy), vascular occlusion, or asymmetric inflammation.

Respiratory-Vascular Coupling Factor (RVCF): Quantifies respiratory modulation of vascular dynamics, reflecting respiratory sinus arrhythmia (RSA)—the phenomenon of heart rate acceleration during inspiration and deceleration during expiration. RVCF is computed as the Pearson correlation coefficient between:
Respiratory-frequency envelope of impedance signal (0.15-0.5 Hz), reflecting respiratory-driven blood volume changes
Acoustic spectral power in the same frequency band

High RVCF (>0.6) indicates intact respiratory-vagal coupling. Low RVCF (<0.3) indicates autonomic dysfunction or respiratory pathology.

### B. Metabolic and Disease-Specific Indices

Metabolic-Health Index (MHI): A composite score integrating classified VOC patterns with impedance-derived hydration and perfusion status. MHI computation employs a trained neural network taking inputs:
VOC pattern classification outputs (metabolic class probabilities)
Impedance magnitude and phase at multiple frequencies
MECI and VRSI values
Temporal trends

MHI ranges from 0 (severe metabolic derangement) to 1 (optimal metabolic health), with thresholds for specific conditions (e.g., MHI<0.4 with ketoacidosis VOC pattern indicates diabetic emergency).

Gut-Brain Index (GBI): Quantifies gut-brain axis integrity through integration of microbiome-associated VOC signatures with vagal tone markers. GBI computation includes:
Dysbiosis VOC pattern scores (indole, skatole, p-cresol, H₂S ratios)
SCFA-associated volatile patterns
MECI (reflecting vagal tone)
Temporal stability of VOC patterns

Elevated GBI (>0.6) indicates gut-brain axis dysfunction associated with neuroinflammation, mood disorders, and neurodegeneration risk. GBI responds to dietary intervention, probiotic treatment, and vagal stimulation.

Neurodegeneration Index (NDI): Integrates neurodegeneration-associated VOC patterns with autonomic biomarkers for early disease detection and progression monitoring. Inputs include:
Lipid peroxidation aldehyde patterns (AD-associated)
Sebaceous VOC patterns (PD-associated)
MECI trajectory (declining in neurodegeneration)
GBI (elevated in prodromal neurodegeneration)

NDI enables presymptomatic detection (particularly for PD, where VOC changes precede motor symptoms by years), disease staging, and treatment response monitoring.

Epilepsy Susceptibility Index (ESI): Computes seizure probability from pre-ictal VOC patterns and autonomic dynamics. Inputs include:
Pre-ictal VOC signature detection (menthone, isopulegol, linalyl acetate)
MECI dynamics (pre-ictal decline)
VRSI changes (pre-ictal instability)
Temporal pattern matching to prior seizure events

ESI provides 15-45 minute seizure prediction lead time, enabling preemptive intervention.

PTSD Severity Index (PSI): Quantifies PTSD symptom severity and hyperarousal state from autonomic and VOC biomarkers. Inputs include:
Baseline autonomic parameters (reduced MECI, elevated VRSI, reduced RVCF)
Stress VOC pattern intensity (cortisol metabolites, catecholamine byproducts)
Hyperarousal dynamics (acute MECI drops, VRSI spikes)
Inflammatory VOC markers

PSI enables objective symptom tracking, treatment response monitoring, and hyperarousal detection for therapeutic intervention.

Oncological Risk Index (ORI): Integrates cancer-associated VOC patterns with inflammatory and metabolic biomarkers for continuous cancer screening. Inputs include:
Oxidative stress VOC patterns (lipid peroxidation aldehydes)
Tumor metabolism markers
Inflammatory VOC patterns
Autonomic dysfunction indicators

ORI provides risk stratification for cancer screening prioritization and treatment response monitoring.

Sepsis Risk Index (SRI): Integrates sepsis-associated VOC patterns with autonomic dysfunction for early sepsis detection. Inputs include:
Metabolic stress VOCs (acetone, 2-butanone)
Oxidative stress VOCs (pentane, ethane, aldehydes)
Inflammatory VOCs (isoprene)
MECI (declining in sepsis)
VRSI (increasing in sepsis)
Impedance changes (tissue edema)

SRI enables early sepsis detection before clinical deterioration, when intervention is most effective.

Inflammatory Burden Index (IBI): Quantifies systemic inflammation from VOC patterns and autonomic biomarkers. Inputs include:
Inflammatory VOCs (isoprene, pentane, ethane)
Lipid peroxidation products
MECI (inversely correlated with inflammation through cholinergic anti-inflammatory pathway)

IBI tracks inflammatory disease activity, treatment response, and cardiovascular risk.

Respiratory Health Index (RHI): Integrates respiratory-associated VOC patterns with pulmonary function indicators. Inputs include:
Airway inflammation VOCs
Exhaled nitric oxide surrogate markers
RVCF (respiratory-autonomic coupling)
Impedance respiratory modulation

RHI enables COPD, asthma, and respiratory infection monitoring.

Cardiovascular Risk Index (CRI): Composite cardiovascular risk score integrating multiple biomarkers. Inputs include:
VRSI (arterial stiffness)
MECI (autonomic function)
Inflammatory VOC patterns
Metabolic VOC patterns
Heart rate and rhythm indicators

CRI provides continuous cardiovascular risk stratification.

### VOC Detection, Classification, and Pattern Recognition

### A. Principles of VOC Pattern Classification

The present invention employs VOC pattern classification rather than simple concentration measurement of individual compounds. This approach recognizes that disease states produce characteristic VOC spectral fingerprints—specific combinations of multiple compounds at defined concentration ratios—that provide diagnostic information unavailable from any single analyte.

Single-compound detection suffers from fundamental limitations: (1) Many conditions alter the same compounds, reducing specificity; (2) Normal biological variation produces overlapping concentration ranges between healthy and diseased states; (3) Exogenous exposures (diet, environment, medications) confound endogenous biomarker interpretation; (4) Compound-specific sensors require impractical arrays for comprehensive screening.

Pattern classification overcomes these limitations by: (1) Exploiting multi-compound signatures unique to specific conditions; (2) Learning decision boundaries that accommodate normal variation; (3) Incorporating temporal dynamics that distinguish endogenous from exogenous sources; (4) Using sensor arrays with overlapping sensitivity to reconstruct complex mixtures.

### B. MOX Sensor Array Design and Operation

The VOC sensor array comprises multiple metal-oxide semiconductor (MOX) sensor elements with differential chemical sensitivity:

Tin dioxide (SnO₂) sensors: Broad sensitivity to reducing gases including alcohols, aldehydes, ketones, and hydrocarbons. Most widely used MOX material with well-characterized response properties.

Tungsten trioxide (WO₃) sensors: Preferential sensitivity to nitrogen oxides, ammonia, and hydrogen sulfide. Complementary selectivity to SnO₂.

Zinc oxide (ZnO) sensors: Sensitivity to alcohols, hydrocarbons, and aromatic compounds. Different response kinetics than SnO₂.

Indium oxide (In₂O₃) sensors: Sensitivity to oxidizing gases, ozone, and nitrogen dioxide. Enables detection of oxidizing species.

Mixed metal oxides and doped materials: Tailored selectivity through metal additives (Pd, Pt, Au nanoparticles) or composite formulations.

Sensor operation involves heating the metal-oxide material to temperatures where gas-surface reactions occur (typically 200-450° C.). Target gas molecules adsorb onto the surface, undergoing oxidation or reduction reactions that transfer electrons to or from the metal oxide, changing electrical conductivity. Conductivity change magnitude depends on gas concentration, identity, and sensor temperature.

Temperature modulation enhances selectivity. Different gases exhibit characteristic response kinetics at different temperatures. By cycling sensor temperature through programmed profiles (e.g., 200° C.→300° C.→400° C.→300° C.→200° C. over 30-60 seconds), the resulting conductivity transient encodes gas mixture composition in its temporal shape. This “temperature-modulated” or “pulsed temperature” operation produces spectral fingerprints unique to specific mixtures.

### C. Signal Processing and Feature Extraction

Raw sensor signals undergo preprocessing including:
Baseline correction: Compensating for sensor drift and environmental changes
Normalization: Scaling to reference conditions for consistent interpretation
Filtering: Removing noise while preserving relevant dynamics
Temperature compensation: Correcting for ambient temperature effects on sensor response

Feature extraction computes relevant characteristics for classification:

Steady-state features: Sensor resistance/conductance at each temperature setpoint; resistance ratios between sensors; time to reach steady state.

Transient features: Rise time, fall time, and response curve shape during temperature transitions; first and second derivatives; inflection points.

Spectral features: Frequency content of response signals through Fourier transform; wavelet coefficients capturing multi-scale dynamics.

Statistical features: Mean, variance, skewness, kurtosis of response windows; percentiles and quantiles.

### D. Machine Learning Classification Architectures

The processor executes classification algorithms including:

Convolutional Neural Networks (CNNs): Hierarchical feature learning from raw or minimally processed sensor signals. 1D CNNs process temporal sensor response curves; 2D CNNs process time-temperature images. Convolutional layers learn local patterns; pooling layers provide translation invariance; fully connected layers perform classification.

Recurrent Neural Networks (RNNs): Sequential modeling capturing temporal dependencies in sensor responses. Long Short-Term Memory (LSTM) and Gated Recurrent Unit (GRU) architectures handle long-range dependencies and variable-length sequences.

Transformer Networks: Attention-based architectures capturing global relationships in input sequences. Self-attention enables modeling of interactions between distant time points or sensor elements.

Graph Neural Networks (GNNs): Specifically GraphSAGE architecture for modeling relationships between sensor elements and multimodal data sources. Sensor array topology and cross-modal correlations are represented as graph structures.

Model-Agnostic Meta-Learning (MAML): Rapid adaptation to new individuals through meta-learning. MAML learns initialization parameters enabling fast personalization with minimal user-specific data, establishing individual baselines within approximately 7 days of wear. Ensemble Methods: Combining multiple classifiers (random forest, gradient boosting, neural networks) for robust prediction. Ensemble diversity captures different aspects of VOC patterns.

### E. Disease-Specific VOC Pattern Signatures

The classification system recognizes the following validated VOC pattern categories:

### Metabolic Disease Patterns:

Diabetic Ketoacidosis (DKA): Elevated acetone (>2 ppm skin emission) as primary marker; elevated β-hydroxybutyrate-derived volatiles; characteristic acetone/isopropanol ratio distinguishing DKA from fasting ketosis; concurrent reduction in VRSI and MECI indicating metabolic-autonomic stress.

Type 2 Diabetes: Moderately elevated acetone; altered exhaled nitric oxide; elevated methylglyoxal-derived volatiles from advanced glycation; metabolic VOC pattern changes with glycemic control.

Chronic Kidney Disease (CKD): Elevated ammonia, trimethylamine, dimethylamine from impaired renal clearance; elevated p-cresyl sulfate and indoxyl sulfate metabolites; uremic fetor pattern in advanced disease.

Hepatic Dysfunction: Elevated dimethyl sulfide, hydrogen sulfide, and mercaptans (fetor hepaticus); altered limonene metabolism; characteristic hepatic encephalopathy VOC signature. Gut Dysbiosis Patterns:

Inflammatory Bowel Disease (IBD): Elevated indole, skatole, p-cresol; elevated hydrogen sulfide; altered SCFA ratios; inflammatory VOC markers.

Small Intestinal Bacterial Overgrowth (SIBO): Elevated hydrogen and methane; altered carbohydrate fermentation products; malabsorption-associated VOC changes.

Clostridioides difficile Infection: Elevated p-cresol and isocaproic acid; characteristic pattern distinguishing from other diarrheal illnesses.

### Neurological Disease Patterns:

Alzheimer's Disease (AD): Elevated lipid peroxidation aldehydes (hexanal, heptanal, nonanal) reflecting neuronal membrane damage; altered isoprene patterns; inflammatory VOC markers; changes potentially detectable in mild cognitive impairment (MCI) stage.

Parkinson's Disease (PD): Characteristic sebaceous VOC signature (hippuric acid, eicosane, octadecanal) potentially detectable years before motor symptom onset; autonomic dysfunction (reduced MECI) as early marker; altered gut dysbiosis pattern reflecting prodromal GI involvement.

Epilepsy (Pre-ictal): Seizure-associated VOC changes (menthone, isopulegol, linalyl acetate) identified through seizure-alert dog research; pre-ictal autonomic instability (declining MECI, SSR changes); pattern-based prediction 15-45 minutes before seizure onset.

Multiple Sclerosis: Inflammatory VOC markers during relapses; oxidative stress pattern during disease activity.

### Psychiatric Disease Patterns:

PTSD: Chronic stress VOC signatures (cortisol metabolites, catecholamine byproducts); inflammatory markers; autonomic hyperarousal pattern (reduced baseline MECI, elevated VRSI); acute exacerbation signatures.

Major Depressive Disorder: Inflammatory VOC markers; altered neurotransmitter metabolite patterns; reduced MECI reflecting vagal dysfunction.

Schizophrenia: Altered volatile profile including pentane and carbon disulfide; oxidative stress markers.

### Oncological Patterns:

Lung Cancer: Elevated aldehydes (hexanal, heptanal, octanal, nonanal); benzene derivatives; alkanes (pentane, hexane); distinguishing patterns for different histological types.

Colorectal Cancer: Elevated indole derivatives reflecting tumor-microbiome interactions; altered SCFA patterns; oxidative stress markers.

Breast Cancer: Elevated alkanes and methylated alkanes; oxidative stress pattern.

Hepatocellular Carcinoma: Elevated dimethyl sulfide; characteristic hepatic metabolism changes.

Gastric Cancer: Elevated volatile nitriles and alcohols; Helicobacter pylori-associated pattern in subset.

### Infectious Disease Patterns:

Sepsis: Elevated acetone and 2-butanone (metabolic stress); elevated pentane and ethane (oxidative stress); elevated isoprene (inflammatory); declining MECI (autonomic dysfunction); pattern changes hours before clinical deterioration.

Bacterial Pneumonia: Pathogen-specific VOC signatures; inflammatory markers; respiratory pattern changes.

Tuberculosis: Methyl nicotinate and other Mycobacterium-specific volatiles.

COVID-19: Specific breath/skin VOC patterns identified in research; inflammatory markers; pattern changes with disease severity.

Urinary Tract Infection: Elevated ammonia; bacterial metabolism products; inflammation markers.

### Cardiovascular Disease Patterns:

Heart Failure: Elevated acetone (cardiac cachexia); inflammatory markers; declining MECI; fluid overload indicators from impedance.

Acute Coronary Syndrome: Oxidative stress markers during ischemia; inflammatory activation pattern.

Atrial Fibrillation: Autonomic dysfunction pattern; inflammation markers.

### Respiratory Disease Patterns:

COPD: Inflammatory VOCs; oxidative stress markers; exhaled pentane and ethane. Asthma: Exhaled nitric oxide surrogate markers; leukotriene metabolites; airway inflammation pattern.

Pulmonary Fibrosis: Oxidative stress markers; inflammatory pattern.

### Autoimmune and Inflammatory Patterns:

Rheumatoid Arthritis: Elevated pentane and other oxidative stress markers during flares; inflammatory VOC pattern.

Systemic Lupus Erythematosus: Inflammatory markers; oxidative stress pattern.

### Skin-Based Biomarkers and Relationship to Vagal Function

The present invention exploits the intimate physiological relationship between autonomic (vagal) function and skin-based biomarkers. This relationship operates through multiple interconnected pathways that enable diagnostic specificity impossible with either modality alone.

### A. Vagal Regulation of Cutaneous Blood Flow

The vagus nerve modulates cutaneous microcirculation through several mechanisms: Direct parasympathetic vasodilation: Vagal efferents release acetylcholine and vasoactive intestinal peptide (VIP) causing vasodilation in certain vascular beds.

Indirect sympathetic modulation: Vagal activity influences central autonomic centers, modulating sympathetic outflow to cutaneous vessels. High vagal tone is associated with reduced sympathetic vasoconstrictor activity.

Inflammatory modulation: Through the cholinergic anti-inflammatory pathway, vagal activity reduces inflammatory cytokines that cause endothelial dysfunction and alter vascular reactivity. Reduced vagal tone produces sympathetic dominance, causing cutaneous vasoconstriction that alters delivery of blood-borne volatile compounds to the skin surface. The present invention detects these changes through concurrent VRSI (reflecting vascular compliance) and VOC emission measurement. Specifically:

Decreased VRSI combined with reduced VOC emission indicates vasoconstriction-mediated reduction in metabolite delivery to skin, even when systemic VOC levels remain normal—distinguishing peripheral vascular dysfunction from metabolic improvement.

Elevated VRSI combined with increased VOC emission indicates vasodilation with enhanced metabolite delivery—potentially distinguishing systemic metabolic changes from local effects. This coupling enables differentiation of central (metabolic) from peripheral (vascular) causes of VOC pattern changes.

### B. Vagal Influence on Eccrine Sweat Gland Activity

Eccrine sweat glands are innervated primarily by sympathetic cholinergic fibers (unusual sympathetic fibers releasing acetylcholine rather than norepinephrine). However, vagal tone modulates the central autonomic network that regulates sweating:

High vagal tone (elevated MECI) correlates with balanced autonomic function and normal sweat gland responsiveness to thermal and emotional stimuli.

Vagal withdrawal during stress or illness produces sympathetic overdrive that can manifest as hyperhidrosis (excessive sweating from sympathetic activation) or paradoxical anhidrosis (sweat suppression from autonomic exhaustion) depending on chronicity.

The present invention's bio-impedance module detects skin hydration changes reflecting sweat gland activity, while the VOC sensor quantifies sweat-borne metabolites. Correlation analysis between MECI and sweat-VOC emission rates enables differentiation of:

Metabolic changes (altered systemic VOC production with normal sweat delivery): VOC pattern changes without corresponding MECI or hydration changes.

Autonomic changes (altered VOC delivery with normal production): VOC emission changes tracking with MECI and hydration, without proportional VOC pattern changes.

Combined changes: Coordinated VOC pattern and autonomic biomarker changes indicating systemic illness affecting both metabolism and autonomic function.

### C. Inflammatory Coupling Between Vagal Function and Skin VOC Emission

The vagus nerve plays a central role in immune regulation through the cholinergic anti-inflammatory pathway. This pathway operates as follows:
1. Inflammatory stimuli (infection, tissue damage, toxins) activate peripheral immune cells.
2. Cytokines (TNF-α, IL-1B, IL-6) released by activated immune cells enter circulation.
3. Circulating cytokines are sensed by vagal afferent terminals expressing cytokine receptors.
4. Afferent signals reach the nucleus tractus solitarius and activate the inflammatory reflex.
5. Vagal efferent activity increases, releasing acetylcholine at peripheral sites.
6. Acetylcholine binds α7 nicotinic acetylcholine receptors (α7nAChR) on macrophages and other immune cells.
7. α7nAChR activation inhibits NF-κB signaling and suppresses pro-inflammatory cytokine production.

Reduced vagal tone (low MECI) diminishes this anti-inflammatory mechanism, permitting unchecked inflammatory cytokine release. Systemic inflammation alters metabolism through multiple mechanisms:

Hepatic acute phase response changes protein synthesis and metabolic enzyme expression.

Cytokines induce oxidative stress, increasing lipid peroxidation products.

Metabolic rate increases (fever), altering substrate utilization.

Anorexia and catabolismchange nutrient availability.

These metabolic changes produce inflammation-associated VOCs including isoprene (mevalonate pathway product), pentane and ethane (lipid peroxidation), and altered ratios of various compounds.

The present invention detects this coupling by correlating MECI trends with inflammatory VOC markers. A pattern of declining MECI with rising inflammatory VOC concentration indicates vagal withdrawal with consequent inflammatory activation—an early indicator of:
Acute infection and sepsis
Chronic inflammatory diseases (IBD, rheumatoid arthritis, lupus)
Cardiovascular inflammation (atherosclerosis, heart failure)
Neuroinflammation (Alzheimer's, Parkinson's, multiple sclerosis)
Cancer-associated inflammation

### D. Gut-Brain Axis and Vagal Mediation of Microbiome Signals

The vagus nerve serves as the primary neural pathway for gut-brain communication. This bidirectional signaling operates through:

Vagal afferents: Approximately 80% of vagal fibers are afferent (sensory), terminating in the intestinal mucosa where they sense:
Mechanical stimuli (distension, motility)
Chemical stimuli (nutrients, pH, osmolarity)
Microbial metabolites (SCFAs, indoles, bile acid derivatives)
Immune signals (cytokines from mucosal immune cells)
Hormones (CCK, GLP-1, PYY from enteroendocrine cells)

Vagal efferents: Motor fibers modulate:
Gut motility (promoting propulsive contractions)
Secretion (gastric acid, pancreatic enzymes, bile)
Mucosal blood flow
Mucosal immune function (anti-inflammatory signaling)

Intestinal microbiota produce volatile metabolites that serve as signals in this communication: Short-chain fatty acids (SCFAs): Acetate, propionate, and butyrate produced by bacterial fermentation of dietary fiber. SCFAs signal through G-protein-coupled receptors (GPR41, GPR43) on enteroendocrine cells, immune cells, and vagal afferents. SCFA-derived volatiles detectable at skin reflect gut fermentative activity.

Indole derivatives: Indole, skatole, and indole-3-acetate produced by bacterial tryptophan metabolism. Elevated indoles indicate proteolytic fermentation associated with dysbiosis.

Phenolic compounds: Phenol, p-cresol, and 4-ethylphenol from tyrosine metabolism. Associated with gut dysbiosis and uremic toxicity in CKD.

Sulfur compounds: Hydrogen sulfide from sulfate-reducing bacteria; methanethiol from methionine metabolism. Associated with IBD and potentially colorectal cancer.

Bile acid metabolites: Secondary bile acids produced by bacterial modification of primary bile acids. Volatile derivatives may indicate bile acid malabsorption or dysbiosis.

The present invention computes the Gut-Brain Index (GBI) by correlating microbiome-associated VOC patterns with vagal tone (MECI):

$$GBI = f ( Dysbiosis\_VOC \_score , MECI , temporal\_stability )$$

where Dysbiosis_VOC_score reflects pattern classification of gut-derived volatiles, MECI reflects vagal function, and temporal_stability captures pattern consistency over time. Elevated GBI indicates gut-brain axis dysfunction through interpretive patterns: High dysbiosis VOCs+normal MECI: Gut dysbiosis without (yet) affecting vagal function High dysbiosis VOCs+low MECI: Established gut-brain axis dysfunction with vagal impairment

Normal dysbiosis VOCs+low MECI: Vagal dysfunction from non-gut etiology

Improving GBI: Response to dietary intervention, probiotics, or vagal stimulation

### E. Metabolic-Autonomic Feedback Loops

Metabolic derangements detected through skin VOC analysis exert reciprocal effects on vagal function, creating bidirectional relationships the present invention exploits:

### Diabetic Ketoacidosis→Autonomic Dysfunction:

Ketone body accumulation produces metabolic acidosis. Acidosis directly impairs vagal nerve conduction and reduces cardiac vagal modulation. The pattern of elevated acetone combined with declining MECI and VRSI indicates DKA with autonomic compromise, requiring urgent intervention.

### Uremia→Autonomic Neuropathy:

Uremic toxins accumulating in chronic kidney disease (particularly p-cresyl sulfate, indoxyl sulfate, and trimethylamine-N-oxide) have direct neurotoxic effects, damaging vagal neurons and ganglia. Elevated ammonia and uremic VOC markers combined with declining MECI indicates uremic autonomic neuropathy, a serious complication associated with increased cardiovascular mortality.

### Hepatic Encephalopathy→Autonomic Dysfunction:

Hepatic failure produces ammonia and mercaptan accumulation with direct CNS effects. Hepatic VOC pattern combined with declining MECI and confusion indicates hepatic encephalopathy requiring treatment.

### Sepsis→Multi-System Dysfunction:

Sepsis produces metabolic stress (ketogenesis), oxidative stress (lipid peroxidation), and profound autonomic dysfunction (vagal withdrawal). The combination of sepsis-associated VOC patterns with rapidly declining MECI indicates impending decompensation.

### Cancer→Cachexia and Autonomic Dysfunction:

Advanced cancer produces metabolic alterations (Warburg effect, cachexia) with oxidative stress and inflammatory VOC markers. Declining MECI reflects cancer-associated autonomic dysfunction and inflammatory burden.

The present invention detects these bidirectional relationships through multimodal pattern recognition:
Elevated acetone+declining VRSI+declining MECI→DKA with autonomic compromise
Elevated ammonia+uremic pattern+declining MECI→Uremic autonomic neuropathy
Elevated isoprene+inflammatory pattern+declining RVCF→Systemic inflammation with respiratory-autonomic uncoupling
Elevated dysbiosis VOCs+declining MECI+elevated GBI→Gut-brain axis dysfunction
Elevated sepsis VOCs+rapidly declining MECI+impedance changes→Sepsis progression

### F. Circadian Coupling of Vagal Tone and Skin VOC Emission

Both vagal activity and skin VOC emission follow circadian rhythms modulated by the central autonomic network and master circadian clock in the suprachiasmatic nucleus:

Vagal tone circadian pattern: MECI peaks during nocturnal sleep (elevated parasympathetic activity during rest) and nadirs during morning awakening (sympathetic activation for daytime activity). This rhythm is disrupted in:
Shift work and jet lag
Sleep disorders (sleep apnea, insomnia)
Depression and anxiety
Autonomic neuropathy
Heart failure

VOC emission circadian pattern: Acetone emission follows feeding/fasting cycles, peaking during late-night/early-morning fasting when ketogenesis is active. Sebaceous secretion follows diurnal patterns. Inflammatory VOCs follow cortisol rhythm with morning peaks. The present invention's continuous monitoring captures these circadian patterns, enabling detection of:

Circadian disruption: Loss of normal MECI circadian variation combined with flattened VOC circadian rhythms indicates central autonomic dysregulation—a risk factor for cardiovascular disease, metabolic syndrome, and neurodegeneration.

Phase shifts: Altered timing of MECI and VOC rhythms without amplitude loss may indicate circadian misalignment from shift work or jet lag.

Amplitude changes: Reduced circadian amplitude (“flattening”) indicates autonomic rigidity associated with aging, chronic illness, and reduced physiological reserve.

Pattern decoupling: MECI and VOC rhythms normally co-vary; decoupling suggests specific pathology affecting one system.

### G. Stress-Induced Coupling

Acute psychological stress produces a characteristic temporal cascade of autonomic and metabolic changes that the present invention detects:

Immediate phase (seconds): Vagal withdrawal produces rapid MECI decline as parasympathetic inhibition of heart rate is released.

Early phase (tens of seconds): Sympathetic activation produces VRSI increase (vasoconstriction) and electrodermal changes detected through impedance.

Intermediate phase (minutes): Catecholamine and cortisol release alter metabolism, beginning VOC pattern changes. Stress-associated volatiles (cortisol metabolites, catecholamine byproducts) appear.

Sustained phase (hours): Continued stress produces inflammatory activation with inflammatory VOC markers.

Recovery phase: MECI recovery, VRSI normalization, and VOC pattern return to baseline after stressor removal.

This temporal cascade enables differentiation of:

Acute stress: Rapid MECI decline with recovery after stressor removal; brief VOC changes.

Chronic stress: Persistent MECI depression with sustained VOC pattern changes; blunted stress response (reduced dynamic range); elevated inflammatory markers.

Maladaptive response: Exaggerated MECI decline with delayed recovery; excessive VOC changes; pattern consistent with anxiety or PTSD.

Resilient response: Moderate MECI decline with rapid recovery; minimal VOC perturbation. Chronic stress patterns identified through the present invention predict:
Cardiovascular disease risk (sustained sympathetic dominance)
Metabolic syndrome progression (cortisol-driven metabolic changes)
Immunosuppression (reduced anti-inflammatory signaling)
Mood disorder vulnerability (autonomic dysfunction, inflammatory activation)

### Integrated Sensor Architecture

The system architecture integrates four primary sensing modalities in a unified wearable platform:

### A. Bone-Conduction Transducer (Element 101)

A miniaturized piezoelectric actuator configured for bidirectional operation as both acoustic emitter and mechanical receiver. Specifications include:

Physical dimensions: Diameter≤17 mm, thickness≤3 mm, compatible with smartwatch and collar form factors.

Piezoelectric material: Lead zirconate titanate (PZT) or lead-free alternatives (potassium sodium niobate, barium titanate) for biocompatibility.

Frequency range: 32 Hz to 1 MHz, spanning:
Low frequencies (32-300 Hz): Vascular resonance, gross tissue mechanical properties
Mid frequencies (300 Hz-20 kHz): Tissue viscoelasticity, blood flow dynamics
High frequencies (20 kHz-1 MHz): Fine tissue structure, acoustic scattering

### Operating Modes:

Continuous wave: Single-frequency excitation for resonance detection
Frequency sweep: Chirp excitation scanning frequency range for spectral characterization
Pulsed: Impulse excitation for time-of-flight and reflection analysis

Acoustic coupling: Direct skin contact through biocompatible elastomer coupling layer ensuring consistent mechanical interface.

Safety: Acoustic intensity limited to diagnostic ultrasound levels (MI<1.9, TI<1.0); thermal rise<2° C.

### B. Piezoelectric Sensor Array (Element 102)

A secondary piezoelectric element or MEMS accelerometer array positioned to capture transmitted and scattered mechanical waves:

### Sensor Types:

Piezoelectric film (PVDF): High sensitivity, flexibility, biocompatibility
MEMS accelerometer: Digital output, integrated signal conditioning, low power
Piezoelectric ceramic: High sensitivity for specific frequency bands

Bandwidth: DC to 20 kHz minimum; extended to 100 kHz for high-frequency applications.

Sensitivity: Sufficient to detect micron-scale tissue displacements from acoustic excitation.

Positioning: Adjacent to or opposite bone-conduction transducer depending on embodiment (reflection vs. transmission measurement geometry).

Array configurations: Single sensor for basic measurements; 2-4 sensor arrays for spatial resolution and bilateral symmetry assessment.

### C. Tetrapolar Bio-Impedance Module (Element 103)

Four electrodes arranged in tetrapolar (4-electrode) configuration for accurate tissue impedance measurement:

Electrode configuration: Two outer electrodes inject current; two inner electrodes measure voltage. This configuration eliminates electrode-skin contact impedance from measurement.

Electrode materials: Ag/AgCl for gel-coupled measurements; titanium nitride or gold for dry electrodes; conductive polymers for flexible applications.

Electrode spacing: 8-15 mm depending on target tissue depth; closer spacing samples superficial tissues, wider spacing penetrates deeper.

Current injection: Sinusoidal current, ≤10 μA rms at all frequencies (below perception threshold), swept across frequency range.

Frequency range: 10 Hz to 1 MHz, capturing:
Low frequencies (10-1000 Hz): Extracellular fluid, tissue hydration
Mid frequencies (1-100 kHz): Cellular membrane properties
High frequencies (100 kHz-1 MHz): Intracellular properties, blood conductivity

Measurements: Complex impedance magnitude |Z(f)| and phase q(f) at multiple frequencies; derived parameters include resistance (R), reactance (X), phase angle, Cole model parameters.

### D. Metal-Oxide VOC Sensor Array (Element 104)

Multiple MOX sensor elements with differential chemical sensitivity positioned to sample skin VOC emissions:

Sensor configuration: 4-8 individual MOX sensing elements with varied metal-oxide compositions:
SnO₂ (tin dioxide): Broad reducing gas sensitivity
WO₃ (tungsten trioxide): NOx, H₂S, NH₃ sensitivity
ZnO (zinc oxide): Alcohol, hydrocarbon sensitivity
In₂O₃ (indium oxide): Oxidizing gas sensitivity
Doped/composite materials: Application-specific selectivity

Operating temperature: 200-450° C. with temperature modulation for selectivity enhancement. Micro-hotplate MEMS technology enables rapid temperature cycling with minimal power.

Sampling geometry: Sensor chamber positioned within 5 mm of skin surface; optional heated sampling chamber (35-42° C.) enhances VOC emission.

Environmental compensation: Integrated temperature and humidity sensors for baseline correction; ambient air reference sampling.

Power management: Duty-cycled operation (active sampling periods alternating with low-power sleep) extending battery life while maintaining adequate temporal resolution.

### E. Processing Unit (Element 105)

Low-power microcontroller executing signal acquisition, preprocessing, feature extraction, and classification:

Processor architecture: ARM Cortex-M4F or equivalent providing digital signal processing capability with floating-point unit; 100+ MHz clock; 256+ KB RAM; 1+MB flash.

Signal acquisition: Multi-channel simultaneous sampling at appropriate rates (5+kHz for acoustic/impedance; 0.1-10 Hz for VOC).

On-device processing: Preprocessing, feature extraction, and inference for real-time biomarker computation; model parameters stored in flash.

Communication: Bluetooth Low Energy (BLE) 5.0+ for smartphone connectivity; optional cellular (LTE-M/NB-IoT) for standalone operation.

Power management: Adaptive duty cycling; sleep modes; efficient voltage regulation; target 8-24 hour operation on 300-500 mAh battery.

### F. Additional Components

Inertial Measurement Unit (IMU, Element 106): 6-axis accelerometer/gyroscope for motion artifact rejection, activity classification, and postural assessment.

Environmental Sensors (Element 107): Temperature and humidity sensors for compensation algorithms.

Storage (Element 108): 8-32 GB flash memory for extended data logging when communication unavailable.

Power System (Element 109): Rechargeable lithium-polymer battery with wireless charging capability.

### Smartwatch Embodiment (Human Peripheral Monitoring)

FIG. 4 illustrates the smartwatch embodiment optimized for continuous ambulatory wrist monitoring:

Caseback Assembly: The watch caseback integrates sensing elements in skin-contacting surface:
Central piezoelectric actuator (401): 8 mm diameter, positioned over radial artery
Surrounding piezoelectric sensors (402): Ring of 4 sensors for bilateral and spatial resolution
VOC sensor chamber (404): Peripheral chamber with micro-perforated PTFE membrane maintaining IP68 water resistance while permitting gas exchange
Impedance electrodes: Four titanium nitride electrodes in corners of caseback

Watch Case: Houses processing electronics, battery, display, and user interface:
Microcontroller (405): Main processor executing algorithms
Signal conditioning (406): Analog front-end with programmable gain amplifiers
Power management (407): Battery charging, voltage regulation, power sequencing
Data acquisition (408): ADCs, timing, synchronization

Form Factor: Standard smartwatch dimensions (40-46 mm case diameter, 10-14 mm thickness); interchangeable bands; water resistance to 50 m.

User Interface: Touchscreen display showing biomarker trends, alerts, and health insights; companion smartphone application for detailed analysis, history, and healthcare integration.

Computed Indices: VRSI, MECI, BASI, RVCF, MHI, GBI, NDI, ESI, PSI, ORI, SRI, IBI, RHI, CRI.

EEG-TYPE HEADPHONE EMBODIMENT (Cranial Neuro-Vagal Monitoring) FIG. 3 illustrates the EEG-type headphone embodiment optimized for cranial neuro-vagal monitoring with bilateral mastoid sensor placement.

### A. Anatomical Rationale

The mastoid process—the bony prominence behind each ear—provides optimal access for vagal and cerebrovascular monitoring due to several anatomical factors:

Proximity to vagal pathways: The mastoid region overlies the course of the vagus nerve as it exits the jugular foramen and descends through the neck. Acoustic energy transmitted through the mastoid bone couples effectively to adjacent vascular and neural structures.

Temporal bone acoustics: The temporal bone surrounding the mastoid provides excellent acoustic transmission characteristics, enabling bone-conduction interrogation of intracranial vascular structures including the internal carotid artery, vertebral arteries, and cerebral vasculature.

Bilateral symmetry: Symmetric bilateral placement enables computation of the Bilateral Autonomic Symmetry Index (BASI), detecting unilateral pathology such as stroke, carotid stenosis, or unilateral vagal dysfunction.

Minimal soft tissue interference: The thin skin over the mastoid process minimizes acoustic attenuation compared to other body locations, improving signal quality.

### B. Hardware Configuration

The headphone embodiment comprises:

Headband Assembly (301): Adjustable headband constructed from medical-grade materials providing consistent bilateral pressure for acoustic coupling. Spring-loaded pivot mechanisms accommodate varying head sizes while maintaining sensor contact.

Bilateral Mastoid Sensor Pods (302, 303): Each pod contains a bone-conduction transducer (17 mm or less diameter) positioned over the mastoid process, a piezoelectric sensor array for mechanical response detection, tetrapolar impedance electrodes (two drive electrodes, two sense electrodes) for cranial bio-impedance measurement, a MOX VOC sensor chamber sampling periauricular skin emissions, and a temperature sensor for thermal compensation.

Bridge Electronics Housing (304): Dorsal headband section containing the microcontroller and signal processing circuitry, wireless communication module (Bluetooth Low Energy), rechargeable battery providing greater than 12 hour operation, and user interface elements including LED indicators and touch controls.

Optional EEG Integration (305): Conductive electrodes at standard 10-20 system positions (Fp1, Fp2, F7, F8, T3, T4, T5, T6) for concurrent electroencephalography, enabling correlation between autonomic biomarkers and brain electrical activity.

### C. Cranial-Specific Biomarkers

The headphone embodiment computes standard biomarkers (VRSI, MECI, BASI, RVCF) plus cranial-specific indices:

Cerebrovascular Resonance Index (CVRI): Derived from acoustic interrogation of intracranial vessels through the temporal bone window, reflecting cerebral arterial compliance and intracranial pressure dynamics.

Cranial Autonomic Asymmetry (CAA): Enhanced BASI computation exploiting bilateral mastoid placement for sensitive detection of hemispheric differences in autonomic regulation.

Neuro-Vagal Coupling Index (NVCI): When combined with EEG, quantifies correlation between vagal biomarkers (MECI) and cortical activity patterns, relevant for epilepsy monitoring, sleep staging, and cognitive assessment.

### D. Applications

The headphone embodiment addresses applications requiring cranial monitoring including migraine prediction and management through pre-ictal cerebrovascular changes detected via CVRI and autonomic prodrome through MECI decline; traumatic brain injury monitoring for post-concussion autonomic dysfunction tracking and cerebrovascular autoregulation assessment; sleep monitoring providing continuous autonomic staging complementing or replacing traditional polysomnography; cognitive performance assessment through real-time autonomic state monitoring for attention, stress, and cognitive load in occupational and educational settings; epilepsy monitoring combining pre-ictal autonomic changes with optional EEG for comprehensive seizure prediction; and neurodegenerative disease detection with enhanced sensitivity for Alzheimer's disease and Parkinson's disease through cranial VOC sampling and cerebrovascular assessment.

### Canine Wearable Collar Embodiment (Veterinary Monitoring)

FIG. 6 illustrates the canine collar embodiment for veterinary applications.

### A. Anatomical Considerations

The canine cervical anatomy provides favorable sensor positioning. The vagus nerve runs within the carotid sheath at the ventrolateral neck in a relatively superficial and accessible location. The carotid artery is palpable for vascular measurements. The thin cervical skin (3-8 mm thickness) permits effective acoustic transmission. Fur coverage requires consideration in electrode and VOC sensor design, addressed through direct skin contact mounting.

### B. Hardware Configuration

Collar Band (601): Medical-grade silicone or neoprene construction, 25-40 mm width, with adjustable sizing for breed variation including small (25-35 cm circumference), medium (35-50 cm), and large (50-70 cm) configurations.

Bone-Conduction Emitter (602): 17 mm or less diameter transducer positioned at ventrolateral location targeting the carotid sheath containing the vagus nerve and carotid artery.

Piezoelectric Receivers (603): Positioned at 90 degrees or 180 degrees from emitter for transmission measurements through cervical tissues.

Dual MOX VOC Sensors (604, 605): Ventral midline and lateral neck positions for comprehensive sampling of skin-emitted volatile organic compounds including stress-associated volatiles.

Impedance Electrodes (606): Four stainless steel or Ag/AgCl electrodes (4 mm diameter) in tetrapolar configuration with conductive gel interface.

Electronics Housing (607): Dorsal collar position with IP67 waterproof rating, containing microcontroller, wireless communication, and rechargeable battery.

Inertial Measurement Unit (608): 6-axis accelerometer and gyroscope for motion artifact rejection, activity classification, and seizure detection confirmation.

### C. Canine-Specific Biomarkers

VRSI-c (Canine Vascular Resonance Shift Index): Calibrated for canine vascular properties with higher baseline resonance frequencies than humans.

MECI-c (Canine Mechanical-Electrical Coherence Index): Calibrated for canine heart rate range (60-140 bpm) and respiratory rate range (15-30 breaths per minute).

SSR (Stress Spectral Ratio): Computed as the ratio of acoustic power in the 20-80 Hz band to power in the 100-200 Hz band. Values greater than 1.5 indicate calm state; values less than 0.8 indicate anxious state.

CSI (Canine Stress Index): Composite stress indicator integrating autonomic biomarkers with behavioral indicators from IMU data.

ESI-c (Canine Epilepsy Susceptibility Index): Derived from pre-ictal VOC patterns combined with MECI-c dynamics for seizure prediction.

### D. Veterinary Applications

Epilepsy Management: Seizure prediction with sensitivity exceeding 80% and lead time exceeding 25 minutes through classification of pre-ictal VOC patterns combined with autonomic biomarker dynamics; ictal detection through rapid MECI-c changes and motion patterns; post-ictal monitoring of recovery trajectory; rescue medication timing optimization.

Separation Anxiety: Objective stress quantification during owner absence through continuous CSI monitoring enabling behavioral intervention assessment.

Working Dog Performance: Military, police, search-and-rescue, and service dog monitoring for fatigue detection, stress assessment, and optimal work-rest scheduling.

Post-Surgical Recovery: Autonomic stability monitoring, pain indicators through stress biomarkers, and early complication detection.

Chronic Disease Management: Monitoring of heart disease, diabetes, and kidney disease in companion animals through metabolic VOC patterns and autonomic biomarkers.

Specifications: IP67 waterproof rating; greater than 48 hour battery life on 500 mAh cell; less than 60 gram weight; wireless charging capability; Bluetooth Low Energy connectivity to smartphone application.

### Plant Monitoring Embodiment (Agricultural and Environmental Applications)

FIG. 7 illustrates the plant monitoring embodiment for agricultural stress detection and environmental monitoring.

### A. Physiological Basis for Plant Acoustic-Impedance Monitoring

Plants exhibit measurable acoustic and electrical properties that reflect physiological state through multiple mechanisms:

Xylem hydraulics: The water-conducting xylem tissue transmits acoustic waves whose propagation velocity and attenuation depend on xylem tension (water potential), cavitation status, and structural integrity. Drought stress increases xylem tension and promotes cavitation (air embolism formation), producing characteristic acoustic signatures including ultrasonic acoustic emissions from bubble formation.

Cell turgor: Plant cell turgor pressure—the force of cell contents against cell walls—determines tissue mechanical properties. Turgor loss during water stress reduces tissue stiffness, shifting acoustic resonance frequencies downward in a measurable manner.

Bio-impedance: Plant tissue impedance reflects water content, ion concentrations, membrane integrity, and cellular structure. Drought stress reduces tissue water content, increasing impedance magnitude. Pathogen infection alters membrane permeability, changing impedance phase characteristics.

Volatile emissions: Plants emit volatile organic compounds (VOCs) through stomata and cuticle in response to stress. Green leaf volatiles (GLVs) including hexenal, hexenol, and hexenyl acetate are released upon mechanical damage. Terpenoids including isoprene, monoterpenes, and sesquiterpenes are emitted in response to heat stress, herbivory, and pathogen attack. Ethylene emission increases during senescence, fruit ripening, and stress responses.

### B. Hardware Configuration

Stem Collar Assembly (701): Adjustable collar accommodating stem diameters from 5 mm to 50 mm, spanning herbaceous plants to woody vines and branches. Constructed from UV-resistant, weatherproof materials rated for outdoor deployment with IP67 environmental protection. Acoustic Sensing Elements (702, 703): Bone-conduction emitter coupled to stem surface through compliant acoustic coupling gel or elastomer, with piezoelectric receiver positioned 180 degrees opposite (transmission geometry) or adjacent (reflection geometry) to emitter. Operating frequency range optimized for plant tissue spanning 100 Hz to 100 kHz.

Bio-Impedance Electrodes (704): Four stainless steel or silver/silver chloride electrodes in tetrapolar configuration, with conductive gel or hydrogel interface maintaining electrical contact despite stem diameter changes during growth.

VOC Sensor Chamber (705): MOX sensor array positioned in ventilated chamber near leaf axils or stem surface, sampling plant-emitted volatiles with temperature and humidity compensation for outdoor environmental variation.

Environmental Sensors (706): Integrated sensors for ambient temperature, relative humidity, photosynthetically active radiation (PAR), and soil moisture via auxiliary probe connection.

Solar Power Module (707): Integrated photovoltaic panel and rechargeable battery for autonomous outdoor operation without external power connection.

Wireless Communication (708): Low-power wide-area network (LPWAN) connectivity including LoRaWAN and NB-IoT protocols for agricultural deployment across large areas with gateway aggregation.

### C. Plant-Specific Biomarkers

Plant Water Stress Index (PWSI): Derived from acoustic velocity changes in xylem (decreasing with increasing tension), impedance magnitude (increasing with dehydration), and stomatal conductance surrogate from VOC emission rates. Scaled 0 to 1 where 0 indicates fully hydrated and 1 indicates severe water stress.

Cavitation Risk Index (CRI-p): Acoustic detection of xylem cavitation events (ultrasonic acoustic emissions from air bubble formation) combined with xylem tension estimation. Elevated CRI-p indicates imminent hydraulic failure risk requiring irrigation intervention.

Pathogen Stress Index (PSI-p): VOC pattern classification detecting pathogen-associated volatile signatures. Fungal infections produce characteristic sesquiterpenoid profiles. Bacterial infections induce specific defense volatile blends. Combined with impedance changes reflecting tissue damage for comprehensive pathogen detection.

Herbivory Detection Index (HDI): Green leaf volatile (GLV) pattern detection indicating mechanical damage from insect feeding or other herbivory, enabling early pest detection before visible damage accumulates.

Growth Vigor Index (GVI): Integrated assessment of plant physiological status combining water status, photosynthetic capacity inferred from diurnal patterns, and stress volatile absence. Declining GVI indicates developing stress before visible symptoms appear.

Ripeness Index (RI): For fruit-bearing plants, VOC pattern classification detecting ethylene and ester compounds associated with fruit maturation, enabling precision harvest timing for optimal quality.

### D. Agricultural Applications

Irrigation Optimization: Real-time PWSI monitoring across crop fields enables precision irrigation scheduling, reducing water use by 20-40% while maintaining yield. Wireless sensor networks aggregate data for field-scale irrigation management decisions.

Disease Early Detection: PSI-p monitoring detects pathogen infection days to weeks before visible symptoms appear, enabling targeted intervention (fungicide, bactericide application) before disease spreads. Particularly valuable for high-value crops including vineyards and orchards where early detection prevents significant economic losses.

Pest Monitoring: HDI-based herbivory detection enables integrated pest management by identifying pest pressure early, reducing prophylactic pesticide application and associated costs and environmental impact.

Drought Monitoring: Network deployment provides landscape-scale drought stress assessment for agricultural planning, forestry management, and ecological monitoring applications.

Frost Damage Assessment: Post-frost impedance and acoustic measurements assess tissue damage severity, informing management decisions regarding crop viability and replanting needs.

Phenology Tracking: GVI and RI monitoring tracks crop development stages for harvest planning, growth regulator application timing, and climate adaptation research.

Controlled Environment Agriculture: Greenhouse and vertical farm deployment enables precise environmental control optimization based on real-time plant stress indicators.

### E. Environmental and Research Applications

Forest Health Monitoring: Early detection of drought stress, pest infestation (bark beetles, emerald ash borer), and disease (sudden oak death, Dutch elm disease) in forest ecosystems.

Ecological Research: Non-destructive, continuous plant physiological monitoring for ecophysiology studies, climate change impact assessment, and phenology research.

Urban Forestry: Monitoring street trees and urban forest health, detecting irrigation needs and stress before visible decline occurs.

Carbon Cycle Monitoring: Plant VOC emissions contribute to atmospheric chemistry; continuous monitoring supports carbon flux estimation and biosphere-atmosphere interaction research.

### Signal Processing and Machine Learning Architecture

The processing pipeline comprises six integrated stages:
Stage 1—Excitation Control: Digital waveform synthesis generates acoustic excitation patterns including frequency sweeps (linear or logarithmic chirps spanning 32 Hz to 1 MHz), sweep duration (100 ms to 10 seconds depending on resolution requirements), amplitude modulation maintaining safe acoustic levels across the frequency range, and synchronization coordinated with impedance measurement timing.
Stage 2—Data Acquisition: Simultaneous multi-channel sampling comprising acoustic channels at 5 kHz or greater sampling rate for bone-conduction signals, impedance channels synchronized with current injection using phase-sensitive detection, VOC channels at 0.1-10 Hz effective rate during temperature modulation cycles, and environmental channels (temperature, humidity, motion) at appropriate rates.
Stage 3—Preprocessing: Signal conditioning for reliable feature extraction including band-pass filtering removing out-of-band noise, notch filtering for interference rejection, motion artifact detection and removal using IMU correlation, baseline drift correction, environmental compensation, and normalization to consistent reference conditions.
Stage 4—Feature Extraction: Computing classification-relevant characteristics including time-domain features (amplitude, waveform shape, timing relationships), frequency-domain features (spectral power, resonance frequencies, bandwidth), time-frequency features (STFT, wavelet coefficients, Wigner-Ville distribution), cross-modal features (coherence, correlation, phase relationships between modalities), and VOC features (steady-state responses, transient dynamics, temperature-modulated patterns).
Stage 5—Machine Learning Classification and Fusion: Multi-stage classification architecture comprising modality-specific classifiers (separate models for acoustic, impedance, and VOC features), feature-level fusion (concatenating features from all modalities for joint classification), decision-level fusion (combining modality-specific predictions for final decision), temporal modeling (LSTM and GRU networks capturing time-series dynamics), graph neural networks (GraphSAGE modeling relationships between features and modalities), and meta-learning (MAML-based personalization adapting to individual baselines).
Stage 6—Biomarker Computation and Output: Computing and reporting health indices including primary biomarkers (VRSI, MECI, BASI, RVCF from acoustic-impedance analysis), disease indices (MHI, GBI, NDI, ESI, PSI, ORI, SRI, IBI, RHI, CRI from multimodal classification), trend analysis (short-term and long-term trajectories), alert generation (threshold-based and pattern-based alerts), and data packaging formatted for display, storage, and transmission.

### Therapeutic Operation Mode (Closed-Loop Neuromodulation)

The present invention optionally provides closed-loop therapeutic intervention through acoustic neuromodulation.

### A. Trigger Conditions

Therapeutic intervention is triggered by MECI below personalized threshold indicating general autonomic imbalance requiring support, ESI above seizure probability threshold indicating epilepsy intervention need, PSI spike indicating hyperarousal requiring PTSD intervention, SRI elevation indicating sepsis progression requiring alert and supportive intervention, or heart rate or rhythm abnormalities requiring cardiovascular support.

### B. Stimulation Parameters

Frequency range of 40-80 Hz, with 40 Hz (gamma frequency) providing cognitive and anti-inflammatory effects. Waveform options include sinusoidal or pulsed patterns, phase-locked to physiological rhythms. Intensity limited to acoustic pressure of 0.5 MPa or less (mechanical index less than 1.9). Duration is adaptive based on response, with maximum 5-minute sessions. Targeting includes phase-locking to cardiac R-wave or respiratory cycle for enhanced efficacy.

### C. Closed-Loop Control Algorithm

The closed-loop control algorithm comprises continuous monitoring of trigger biomarkers (MECI, ESI, PSI), threshold comparison against personalized limits, stimulation initiation when threshold is crossed, real-time monitoring of biomarker response during stimulation, intensity modulation based on response trajectory, termination when biomarker returns to acceptable range or timeout is reached, and response logging for algorithm refinement through reinforcement learning.

### D. Safety Features

Hardware-enforced maximum acoustic output intensity limits, skin temperature monitoring with automatic shutoff if temperature rise exceeds 2 degrees Celsius, maximum session duration limits preventing overexposure, user override capability for manual stimulation termination, and contraindication detection with automatic disable during detected unsafe conditions.

### BRIEF DESCRIPTION OF THE DRAWINGS

FIG. 1 is a system-level architecture block diagram illustrating the multimodal wearable monitoring system comprising a bone-conduction transducer (101), a piezoelectric sensor array (102), tetrapolar impedance electrodes (103), a MOX VOC sensor array (104), a processing unit (105), and a communication interface (106). The diagram shows signal flow from sensors through preprocessing, feature extraction, machine learning classification, biomarker computation, and output generation.

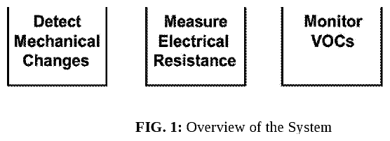

FIG. 2 is a cross-sectional anatomy diagram showing sensor positioning relative to the radial artery, vagal pathways, and skin layers for the wrist smartwatch embodiment. The diagram illustrates acoustic coupling through skin and subcutaneous tissue to underlying vascular structures, electrode placement for bio-impedance measurement, and VOC sensor chamber positioning relative to skin surface.

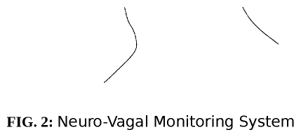

FIG. 3 is an illustration of the EEG-type headphone embodiment showing the headband assembly (301), bilateral mastoid sensor pods (302, 303), bridge electronics housing (304), and optional EEG electrode positions (305). The diagram depicts bilateral mastoid sensor placement for cranial neuro-vagal monitoring with symmetric positioning enabling BASI computation.

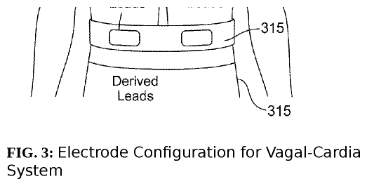

FIG. 4 is an exploded view of the smartwatch embodiment showing caseback sensor integration including piezoelectric actuator (401), piezoelectric sensor (402), impedance electrodes (403), VOC sensor chamber (404), and case electronics including microcontroller (405), signal conditioning amplifier (406), power management (407), and wireless communication module (408). The diagram illustrates component arrangement within smartwatch form factor.

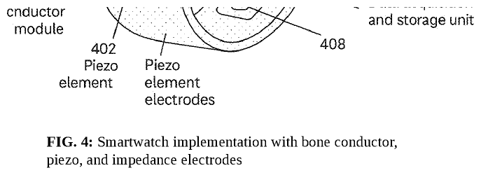

FIG. 5 is a signal processing and classification pipeline flowchart depicting the six processing stages from sensor data acquisition through excitation control, data acquisition, preprocessing, feature extraction, machine learning classification with multimodal fusion, and biomarker index computation and output. The diagram shows parallel processing paths for acoustic, impedance, and VOC modalities converging at the fusion stage.

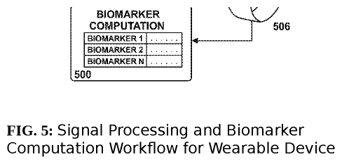

FIG. 6 is an illustration of the canine collar embodiment showing the collar band (601), bone-conduction emitter (602), piezoelectric receivers (603), dual MOX VOC sensors (604, 605), impedance electrodes (606), electronics housing (607), and inertial measurement unit (608). The diagram depicts ventrolateral sensor positioning targeting the cervical vagus nerve and carotid artery.

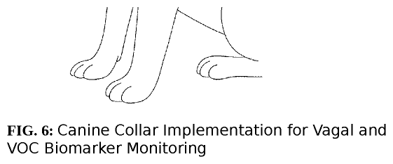

FIG. 7 is an illustration of the plant monitoring collar embodiment showing the stem collar assembly (701), acoustic emitter (702), acoustic receiver (703), bio-impedance electrodes (704), VOC sensor chamber (705), environmental sensors (706), solar power module (707), and wireless communication antenna (708). The diagram depicts sensor positioning around a plant stem for agricultural monitoring.

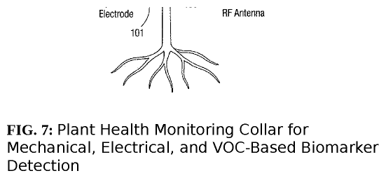

FIG. 8 is a multi-organ vagal resonance pathway diagram illustrating acoustic-mechanical coupling from bone-conduction transducer through tissue to vascular and neural structures. The diagram shows the vagus nerve pathway from brainstem through neck to thoracic and abdominal viscera, with annotation of measurement points accessible through different embodiments.

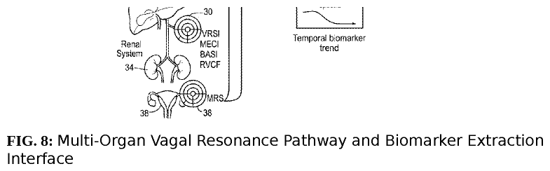

FIG. 9 is a machine learning architecture diagram showing modality-specific feature extraction branches for acoustic, impedance, and VOC data, followed by multimodal fusion using GraphSAGE graph neural network architecture, and classification heads for different disease categories and biomarker indices. The diagram illustrates the hierarchical processing and fusion approach.

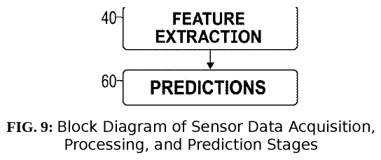

### Data Security and Interoperability

### A. Security Measures

Encryption using AES-256 standard for all stored data and wireless transmission. Authentication through secure pairing protocols for device-to-smartphone communication. Privacy protection through on-device processing minimizing cloud data exposure. Anonymization options for de-identification when sharing research data.

### B. Interoperability Standards

HL7 FHIR (Fast Healthcare Interoperability Resources) compliance for healthcare data exchange enabling EHR integration with major systems including Epic and Cerner. ISO/IEEE 11073 personal health device communication standards for medical device interoperability. HIPAA compliance for United States healthcare privacy regulations. GDPR compliance for European data protection requirements. Apple HealthKit and Google Fit integration for consumer health platform connectivity.

### EXAMPLES OF OPERATION

### Example 1: Diabetic Ketoacidosis Detection and Management

A 52-year-old male with Type 2 diabetes wears the smartwatch embodiment continuously. During an overnight fast following a missed insulin dose, the VOC sensor detects gradually rising acetone (0.8 to 2.1 ppm skin emission over 6 hours). The VOC classifier detects DKA pattern emergence characterized by acetone combined with β-hydroxybutyrate metabolite ratio characteristic of ketoacidosis rather than fasting ketosis. VRSI declines from 0.95 to 0.86 indicating vascular changes. MECI drops from 0.78 to 0.58 indicating autonomic compromise from acidosis. MHI reaches 0.41, crossing the alert threshold. The system generates an urgent alert recommending immediate glucose check and medical attention. The user checks capillary glucose (287 mg/dL) and takes corrective insulin, preventing progression to severe acidosis and emergency department visit.

### Example 2: Canine Seizure Prediction

A 6-year-old Labrador Retriever with idiopathic epilepsy wears the canine collar embodiment. The VOC classifier detects emerging pre-ictal pattern (menthone, isopulegol signature). MECI-c declines from 0.78 to 0.61. SSR inverts from 1.6 to 0.8. ESI-c reaches 0.85 (threshold 0.65). Alert is sent to handler's smartphone 25 minutes before seizure onset. The handler moves the dog to a safe padded area and prepares diazepam. The 25-minute warning enables preparation, environmental safety measures, and rapid response, preventing injury.

### Example 3: PTSD Hyperarousal Detection and Therapeutic Intervention

A 32-year-old combat veteran with PTSD wears the smartwatch with therapeutic stimulation capability enabled. An unexpected loud noise triggers startle response. MECI drops rapidly from 0.62 to 0.48. VRSI spikes from 0.96 to 1.14. Stress VOC pattern emerges (cortisol metabolites, catecholamine byproducts). PSI reaches hyperarousal threshold (0.60). The system initiates closed-loop intervention with 40 Hz acoustic stimulation phase-locked to respiratory cycle. MECI recovers to 0.60 within 4 minutes. PSI returns below threshold. Stimulation terminates. The automated intervention prevents full panic episode and is logged for therapy review with clinician.

### Example 4: Early Sepsis Detection in Post-Surgical Patient

A 68-year-old female recovering from abdominal surgery wears the smartwatch for post-discharge monitoring. On post-operative day 4, subtle VOC pattern changes are detected with acetone slightly elevated and isoprene increasing. SRI rises from 0.28 to 0.52. MECI declines from 0.65 to 0.52. Impedance changes suggest developing tissue edema. SRI reaches alert threshold (0.60) and the system generates an alert recommending medical evaluation, also transmitted to the surgical team. The patient is evaluated in clinic where temperature is 38.2° C. and WBC is elevated. Abdominal CT reveals developing abscess. Abscess drainage and antibiotics are initiated. VOC pattern and autonomic biomarkers detected sepsis 12+ hours before fever and clinical deterioration, enabling early intervention that prevented ICU admission.

### Example 5: Gut Dysbiosis Detection and Intervention Monitoring

A 45-year-old male with irritable bowel syndrome symptoms wears the smartwatch. Initial assessment shows GBI elevated at 0.68 with dysbiosis VOC pattern detected (elevated indole/skatole ratio of 2.4 versus normal less than 1.5, reduced SCFA-associated volatiles). MECI is mildly reduced at 0.66 versus expected 0.75. Based on findings, physician recommends dietary modification and targeted probiotic. Over 8 weeks, indole/skatole ratio declines to 1.5, GBI improves to 0.44, and MECI improves to 0.73. Objective monitoring demonstrates treatment response and guides intervention continuation.

### Example 6: Early Parkinson's Disease Detection

A 58-year-old male with subtle resting tremor undergoes routine wellness monitoring. Over 3 months, VOC classifier intermittently detects PD-associated sebaceous signature pattern with increasing confidence (0.45 to 0.65). MECI is slightly below expected for age (0.64 versus 0.70). GBI is mildly elevated (0.52) suggesting prodromal gut involvement. NDI computed at 0.65 reaches advisory threshold. The system generates advisory recommending neurological evaluation. Neurologist notes subtle bradykinesia. DAT-SPECT imaging reveals reduced dopamine transporter binding consistent with early Parkinson's disease. Patient is enrolled in clinical trial for disease-modifying therapy with NDI tracked longitudinally as objective outcome measure.

### Example 7: Lung Cancer Screening Detection

A 62-year-old female with 30 pack-year smoking history wears the smartwatch as part of cancer screening program. At month 4, lung cancer-associated VOC pattern emerges (elevated hexanal, heptanal, benzene derivative changes). ORI reaches 0.60 screening threshold. The system generates advisory recommending medical evaluation and imaging. Low-dose CT chest reveals 12 mm spiculated nodule. PET is positive. Biopsy confirms stage IA non-small cell lung cancer. Surgical resection with curative intent is performed. VOC pattern detection identified early-stage cancer amenable to curative treatment.

### Example 8: Plant Drought Stress Management

A vineyard deploys the plant monitoring collar embodiment across 500 vines. During a heat wave, PWSI rises from baseline 0.2 to 0.6 across the eastern section while western section remains at 0.3. CRI-p begins rising in eastern vines indicating cavitation risk. The wireless network aggregates data to irrigation controller. Targeted irrigation is applied to eastern section only, reducing water use by 35% compared to whole-field irrigation while preventing drought damage. Yield quality is maintained through precision water management.

### Example 9: Forest Pathogen Early Detection

A research forest deploys the plant monitoring collar on sentinel trees. PSI-p rises on three oak trees, detecting sesquiterpenoid pattern consistent with Phytophthora infection. Impedance changes indicate developing tissue damage. Alert is generated 3 weeks before visible symptoms appear. Forest managers isolate affected area and apply targeted treatment, preventing spread to adjacent trees. Early detection through VOC pattern classification enables intervention before pathogen establishment.

### Example 10: Migraine Prediction with EEG Headphone Embodiment

A 35-year-old female with chronic migraine wears the EEG headphone embodiment during prodrome-prone periods. CVRI shows subtle cerebrovascular changes (increased resonance frequency indicating vasoconstriction). MECI declines from 0.72 to 0.65. Optional EEG shows increased theta activity. The system detects migraine prodrome pattern 45 minutes before typical headache onset. Alert enables early abortive medication administration. Triptan taken during prodrome provides complete headache prevention, whereas medication taken after headache onset typically provides only partial relief.

### Advantages of the Invention

1. VOC Pattern Classification Enabling Comprehensive Disease Detection: Beyond simple concentration measurement of individual compounds, the invention employs machine learning classification of multi-dimensional VOC spectral fingerprints. This enables detection of complex conditions characterized by multi-compound patterns rather than single-analyte elevation. Pattern classification achieves 40-60% accuracy improvement over single-compound detection for complex conditions including gut dysbiosis, neurodegeneration, pre-ictal states, sepsis, and cancer.

2. Multimodal Fusion Dramatically Reducing False Positives: Integration of acoustic, electrical, and classified VOC sensing requires concordance across independent measurement channels before generating clinical alerts, achieving approximately 75% false positive reduction compared to any single modality alone. This addresses the critical clinical problem of alert fatigue.

3. Direct Autonomic Measurement Rather Than Surrogate Endpoints: Unlike heart rate variability analysis requiring 1-5 minute stationary measurement windows and providing only downstream cardiac effects, the acoustic-impedance approach directly interrogates vagal-mediated vascular dynamics with beat-to-beat resolution.

4. Continuous Non-Invasive Metabolic Monitoring Without Consumables: Transcutaneous VOC detection operates continuously without blood sampling, finger-pricks, or consumable sensors, eliminating ongoing costs and compliance barriers.

5. Early Disease Detection Before Clinical Manifestation: Pre-symptomatic detection windows include sepsis (12+ hours before clinical deterioration), epileptic seizures (15-45 minutes before onset), Parkinson's disease (potentially years before motor symptoms), and cancer (months before symptomatic presentation).

6. Sepsis Detection Addressing Major Unmet Clinical Need: Integration of sepsis-associated VOC patterns with autonomic dysfunction biomarkers enables early sepsis detection before clinical deterioration, potentially reducing the 11 million annual sepsis deaths worldwide.

7. Cancer Screening Through Continuous Monitoring: The Oncological Risk Index integrates oxidative stress markers, tumor metabolism byproducts, and inflammatory patterns for continuous cancer risk stratification.

8. Comprehensive Neurological and Psychiatric Monitoring: Unified monitoring for Alzheimer's disease, Parkinson's disease, epilepsy, PTSD, and other neurological conditions through disease-specific indices (NDI, ESI, PSI).

9. Gut-Brain Axis Assessment: The Gut-Brain Index provides first-of-kind continuous non-invasive assessment of gut-brain axis function through integration of microbiome-associated VOC patterns with vagal tone biomarkers.

10. Closed-Loop Therapeutic Intervention: Integration of diagnostic sensing with therapeutic neuromodulation enables automated intervention without separate therapeutic devices.

11. Cross-Species and Cross-Kingdom Applicability: The unified sensing framework applies to humans, animals, and plants with calibration adjustments rather than complete redesign, enabling efficient development across biomedical, veterinary, and agricultural markets.

12. Personalized Baseline Adaptation: Model-Agnostic Meta-Learning enables rapid individual baseline establishment within approximately 7 days, improving anomaly detection sensitivity approximately 3-fold compared to population thresholds.

13. Agricultural Applications Through Plant Monitoring: The plant embodiment enables precision agriculture including irrigation optimization reducing water use by 20-40%, disease early detection weeks before visible symptoms, and pest monitoring enabling integrated pest management.

14. Superior Motion Artifact Resilience: Bone-conduction acoustic coupling maintains greater than 90% signal quality during walking, exercise, and occupational activities compared to greater than 40% signal loss in photoplethysmography-based systems.

### INDUSTRIAL APPLICABILITY

A. Consumer Health and Wellness: The global wearable device market exceeded $50 billion in 2024. Consumer applications include stress monitoring, sleep optimization, metabolic wellness, gut health monitoring, and cognitive wellness.

B. Clinical Diagnostics and Remote Patient Monitoring: The remote patient monitoring market is projected to reach $175 billion by 2028. Clinical applications include diabetes management, chronic kidney disease monitoring, post-surgical recovery monitoring, and cardiac rehabilitation.

C. Sepsis Detection and Critical Care: Sepsis represents a $27+ billion annual healthcare burden in the United States. Applications include post-surgical surveillance, immunocompromised patient monitoring, and nursing home surveillance.

D. Oncology and Cancer Screening: Applications include lung cancer screening in high-risk populations, colorectal cancer detection, treatment response monitoring, and recurrence detection.

E. Neurology: Applications include epilepsy management (50+ million affected worldwide), Parkinson's disease early detection (10+ million affected), Alzheimer's disease screening, and post-stroke assessment.

F. Psychiatry and Mental Health: Applications include PTSD assessment (7% adult prevalence), anxiety quantification, depression biomarker tracking, and stress-related disorder monitoring.

G. Gastroenterology: Applications include inflammatory bowel disease monitoring (3+ million US patients), irritable bowel syndrome assessment (25-45 million US patients), and microbiome intervention monitoring.

H. Veterinary Medicine: The global animal health market exceeds $230 billion. Applications include canine epilepsy management, separation anxiety assessment, working dog optimization, and companion animal wellness.

I. Agriculture and Plant Science: The precision agriculture market is projected to reach $15 billion by 2028. Applications include irrigation optimization, disease early detection, pest monitoring, and phenology tracking.

J. Environmental Monitoring: Applications include forest health monitoring, ecological research, urban forestry, and carbon cycle monitoring.

K. Pharmaceutical Research: Applications include clinical trial endpoint biomarkers, treatment response monitoring, and companion diagnostic development.

L. Occupational Health: The occupational health technology market exceeds $8 billion. Applications include fatigue detection, stress monitoring, and fitness-for-duty assessment.

1. A multimodal wearable system for continuous physiological and metabolic monitoring comprising:
(a) a bone-conduction transducer configured to emit frequency-swept acoustic energy through biological tissue spanning frequencies from 32 Hz to 1 MHz and to receive reflected mechanical responses;
(b) a piezoelectric or micro-electromechanical systems (MEMS) sensor coupled to the bone-conduction transducer for detecting mechanical vibrations and tissue resonance characteristics;
(c) a tetrapolar bio-impedance module comprising four electrodes configured to inject alternating micro-currents and measure complex tissue impedance magnitude and phase;
(d) a metal-oxide semiconductor (MOX) volatile organic compound (VOC) sensor array comprising a plurality of sensor elements with differential chemical sensitivity, positioned to detect volatile organic compound emissions from a skin surface of a user;
(e) a processor configured to:
(i) execute time-frequency analysis of acoustic and impedance data to generate autonomic and vascular biomarkers including a Vascular Resonance Shift Index (VRSI) and a Mechanical-Electrical Coherence Index (MECI);
(ii) perform machine learning classification of responses from the MOX VOC sensor array to identify disease-associated volatile organic compound patterns; and
(iii) fuse acoustic data, impedance data, and VOC data using deep learning architectures to compute disease-specific health indices; and
(f) a communication interface for transmitting processed biomarkers and health assessments to an external device;
wherein the bone-conduction transducer, the tetrapolar bio-impedance module, and the MOX VOC sensor array operate concurrently to provide continuous, non-invasive measurement of autonomic function and metabolic state.

2. The system of claim 1, wherein the processor computes one or more disease-specific indices selected from the group consisting of: a Gut-Brain Index (GBI) derived from microbiome-associated VOC signatures correlated with vagal tone; a Neurodegeneration Index (NDI) derived from VOC patterns associated with Alzheimer's disease and Parkinson's disease; an Epilepsy Susceptibility Index (ESI) derived from pre-ictal VOC patterns providing seizure prediction with a lead time of 15 to 45 minutes; a PTSD Severity Index (PSI) derived from stress-associated VOC patterns combined with autonomic hyperarousal markers; a Sepsis Risk Index (SRI) derived from sepsis-associated VOC patterns combined with a declining MECI; an Oncological Risk Index (ORI) derived from cancer-associated VOC patterns; a Metabolic-Health Index (MHI) integrating VOC classification outputs with impedance-derived hydration and perfusion status; a Respiratory Health Index (RHI) integrating airway inflammation markers with respiratory-autonomic coupling; a Cardiovascular Risk Index (CRI) integrating VRSI, MECI, and inflammatory VOC patterns; and an Inflammatory Burden Index (IBI) derived from inflammatory VOC markers.

3. The system of claim 1, wherein the plurality of sensor elements of the MOX VOC sensor array comprises sensor elements selected from the group consisting of: tin dioxide (SnO₂), tungsten trioxide (WO₃), zinc oxide (ZnO), indium oxide (In₂O₃), and mixed metal oxide formulations, and wherein the processor performs temperature modulation of the MOX VOC sensor array cycling through programmed temperature profiles to generate spectral fingerprints encoding VOC mixture composition.

4. The system of claim 1, wherein the machine learning classification comprises at least one architecture selected from the group consisting of: convolutional neural networks (CNN), recurrent neural networks (RNN), long short-term memory networks (LSTM), transformer networks, graph neural networks including GraphSAGE, and Model-Agnostic Meta-Learning (MAML) for personalized baseline adaptation within approximately seven days of continuous wear.

5. The system of claim 1, wherein the system is integrated into a smartwatch form factor comprising a caseback assembly housing the bone-conduction transducer, the piezoelectric or MEMS sensor, the four electrodes of the tetrapolar bio-impedance module, and a VOC sensor chamber containing the MOX VOC sensor array, wherein the VOC sensor chamber includes a micro-perforated membrane permitting gas exchange while maintaining water resistance to at least 50 meters.

6. The system of claim 1, further comprising a closed-loop therapeutic module wherein a detected autonomic dysfunction or an elevated disease-specific index triggers acoustic neuromodulation stimulation at frequencies between 40 Hz and 80 Hz phase-locked to a cardiac cycle or a respiratory cycle, with safety limits comprising an acoustic pressure not exceeding 0.5 MPa, a tissue temperature rise less than 2° C., and an automatic timeout not exceeding 5 minutes, wherein stimulation parameters are optimized through reinforcement learning based on measured biomarker responses.

7. The system of claim 1, wherein the processor further computes a Bilateral Autonomic Symmetry Index (BASI) from bilateral sensor positions to detect unilateral neural deficits, stroke, or asymmetric vascular pathology, and a Respiratory-Vascular Coupling Factor (RVCF) representing a correlation between respiratory-frequency envelope signals and acoustic spectral power reflecting intact respiratory-vagal coupling.

8. The system of claim 1, wherein the system is implemented in a headband assembly configured to position bilateral sensor pods at mastoid process locations, each sensor pod comprising the bone-conduction transducer, the piezoelectric sensor, the tetrapolar bio-impedance electrodes, and the MOX VOC sensor array, wherein the processor computes a Cerebrovascular Resonance Index (CVRI) derived from acoustic interrogation of intracranial vessels through the temporal bone reflecting cerebral arterial compliance, and optionally integrates electroencephalography (EEG) electrodes to compute a Neuro-Vagal Coupling Index (NVCI) for epilepsy monitoring, sleep staging, migraine prodrome detection, and cognitive performance assessment.

9. The system of claim 1, wherein encrypted data transmitted by the communication interface conform to HL7 FHIR and ISO/IEEE 11073 standards for interoperability with electronic health records, and wherein the processor is further configured to detect circadian rhythm disruption by monitoring temporal patterns of the Mechanical-Electrical Coherence Index and volatile organic compound emission over a continuous period exceeding 24 hours, wherein loss of normal nocturnal MECI elevation combined with flattening of VOC circadian amplitude indicates central autonomic dysregulation.

10. A method for detecting disease states through volatile organic compound pattern classification comprising:
(a) continuously sampling skin-emitted volatile organic compounds using a metal-oxide semiconductor sensor array having a plurality of sensor elements with differential chemical sensitivity;
(b) generating multi-dimensional sensor response patterns through temperature modulation of the metal-oxide semiconductor sensor array;
(c) extracting features from the multi-dimensional sensor response patterns, wherein the features include steady-state responses, transient dynamics, and spectral characteristics;
(d) classifying the extracted features using a trained machine learning model to identify disease-associated volatile organic compound patterns;
(e) concurrently measuring autonomic biomarkers including a Vascular Resonance Shift Index (VRSI) and a Mechanical-Electrical Coherence Index (MECI) using bone-conduction acoustic sensing and bio-impedance spectroscopy;
(f) fusing the classified disease-associated volatile organic compound patterns with the autonomic biomarkers to compute one or more disease-specific health indices; and
(g) generating an alert when at least one of the one or more disease-specific health indices exceeds a predetermined threshold.

11. The method of claim 10, wherein the disease-associated volatile organic compound patterns comprise one or more metabolic disease patterns including: a diabetic ketoacidosis pattern characterized by elevated acetone exceeding 2 ppm skin emission and an elevated β-hydroxybutyrate ratio; a chronic kidney disease pattern characterized by elevated ammonia, elevated trimethylamine, and elevated dimethylamine; and a hepatic dysfunction pattern characterized by elevated dimethyl sulfide and elevated mercaptans.

12. The method of claim 10, wherein the disease-associated volatile organic compound patterns comprise one or more neurological disease patterns including: an Alzheimer's disease pattern characterized by elevated lipid peroxidation aldehydes including hexanal, heptanal, and nonanal; a Parkinson's disease pattern characterized by sebaceous VOC signatures including hippuric acid, eicosane, and octadecanal detectable prior to motor symptom onset; and a pre-ictal epilepsy pattern characterized by menthone, isopulegol, and linalyl acetate signatures providing seizure prediction 15 to 45 minutes before onset.

13. The method of claim 10, wherein the disease-associated volatile organic compound patterns comprise one or more oncological patterns including: a lung cancer pattern characterized by elevated aldehydes and elevated benzene derivatives; a colorectal cancer pattern characterized by elevated indole derivatives; and a breast cancer pattern characterized by elevated alkanes and elevated methylated alkanes.

14. The method of claim 10, wherein the disease-associated volatile organic compound patterns comprise one or more infectious and inflammatory disease patterns including: a sepsis pattern characterized by elevated acetone, elevated 2-butanone, elevated isoprene, elevated pentane, and elevated ethane reflecting metabolic stress, oxidative stress, and inflammatory activation combined with a declining MECI indicating autonomic dysfunction; and inflammatory disease patterns including elevated isoprene, elevated pentane, and elevated ethane combined with a declining MECI reflecting an impaired cholinergic anti-inflammatory pathway.

15. The method of claim 10, further comprising: detecting when at least one disease-specific index selected from an Epilepsy Susceptibility Index (ESI), a PTSD Severity Index (PSI), and a Sepsis Risk Index (SRI) exceeds a personalized threshold established through Model-Agnostic Meta-Learning; automatically initiating acoustic neuromodulation stimulation at frequencies between 40 Hz and 80 Hz phase-locked to an endogenous physiological rhythm in response to the detecting; monitoring a biomarker response during stimulation; and modulating stimulation parameters through reinforcement learning based on the biomarker response until the disease-specific index returns below the personalized threshold or a safety timeout is reached.

16. A multi-species physiological monitoring system for continuous assessment of a non-human subject, comprising:
(a) a bone-conduction transducer configured to emit frequency-swept acoustic energy through biological tissue or plant tissue of the non-human subject spanning frequencies from 32 Hz to 1 MHz and to receive reflected mechanical responses;
(b) a tetrapolar bio-impedance module comprising four electrodes configured to measure complex tissue impedance magnitude and phase at a body surface or stem surface of the non-human subject;
(c) a metal-oxide semiconductor (MOX) volatile organic compound (VOC) sensor array positioned to continuously detect and classify volatile organic compound patterns emitted from a skin, integument, or plant surface of the non-human subject;
(d) a processor configured to compute species-calibrated or plant-calibrated autonomic or physiological biomarkers from the acoustic, impedance, and VOC measurements and to perform machine learning classification of VOC patterns to identify disease-associated, stress-associated, or physiological-stress-associated signatures; and
(e) a wearable or attachable housing configured for secure attachment to the non-human subject during normal activity, maintaining continuous sensor contact with target tissue.

17. The multi-species physiological monitoring system of claim 16, wherein the non-human subject comprises a canine, and wherein the housing comprises a collar band configured for cervical attachment positioning the bone-conduction transducer at a ventrolateral cervical location targeting the carotid sheath containing the vagus nerve and carotid artery, and wherein the processor computes a Canine Stress Index (CSI) integrating species-calibrated autonomic biomarkers with behavioral indicators from an inertial measurement unit and a Stress Spectral Ratio (SSR) defined as a ratio of acoustic power in the 20 Hz to 80 Hz band to acoustic power in the 100 Hz to 200 Hz band, enabling operational fitness assessment for military, law enforcement, search-and-rescue, and service canines.

18. The multi-species physiological monitoring system of claim 16, wherein the non-human subject comprises a bovine, equine, or ovine animal, and wherein the processor computes one or more livestock health indices selected from the group consisting of: a metabolic stress index derived from ketone-associated VOC patterns indicating subclinical ketosis or pregnancy toxemia; a mastitis risk index derived from inflammatory VOC patterns combined with a declining Mechanical-Electrical Coherence Index; a lameness early detection index derived from asymmetric autonomic biomarker patterns from bilateral sensor positions; and a reproductive cycle index derived from hormone-associated VOC pattern changes indicating estrus, pregnancy, or parturition proximity, enabling precision livestock health management and early veterinary intervention.

19. The multi-species physiological monitoring system of claim 17, further comprising a Canine Epilepsy Susceptibility Index (ESI-c) computed by the processor from pre-ictal volatile organic compound patterns comprising menthone, isopulegol, and linalyl acetate signatures combined with a declining species-calibrated Mechanical-Electrical Coherence Index, wherein the system generates a seizure prediction alert with a lead time exceeding 20 minutes and sensitivity exceeding 80%, enabling handler preparation, environmental safety measures, and administration of rescue medication prior to seizure onset, and wherein the system further monitors post-ictal autonomic recovery trajectory.

20. The multi-species physiological monitoring system of claim 16, wherein the non-human subject comprises a plant and the housing comprises a stem collar assembly configured for attachment around a plant stem, and wherein the processor computes plant physiological indices comprising: a Plant Water Stress Index (PWSI) derived from acoustic velocity changes in xylem tissue and impedance magnitude changes reflecting dehydration, scaled from 0 indicating fully hydrated to 1 indicating severe water stress; a Cavitation Risk Index (CRI-p) derived from acoustic detection of xylem cavitation events indicating imminent hydraulic failure requiring irrigation intervention; a Pathogen Stress Index (PSI-p) derived from VOC pattern classification detecting fungal or bacterial pathogen-associated volatile signatures days to weeks before visible symptoms; and a Ripeness Index (RI) for fruit-bearing plants derived from ethylene and ester VOC pattern classification enabling precision harvest timing, wherein the system optionally incorporates a solar power module and low-power wide-area network communication for autonomous deployment across agricultural sensor networks.
